(19) United States
(12) Patent Application Publication
(10) Pub. No.: US 2011/0009872 A1
(43) Pub. Date: Jan. 13, 2011

(54) IMPLANT DELIVERY DEVICE AND METHOD

(75) Inventors: Vanisha Mistry, Cambridge (GB); Tim Mead, Cambridge (GB); Andrew Lynn, Cambridge (GB)

Correspondence Address: SUGHRUE MION, PLLC, 2100 PENNSYLVANIA AVENUE, N.W., SUITE 800, WASHINGTON, DC, 20037, US

(73) Assignee: TIGENIX LIMITED, Cambridge (GB)

(21) Appl. No.: 12/740,085

(22) PCT Filed: Oct. 27, 2008
(86) PCT No.: PCT/GB2008/003610

§ 371 Date: Sep. 8, 2010

(30) Foreign Application Priority Data
Oct. 29, 2007 (GB) 0721158.4
Feb. 26, 2008 (GB) 0803449.8
Aug. 22, 2008 (GB) 0815438.7

(51) Int. Cl. A61B 17/56

(52) U.S. Cl. 606/99

(57) ABSTRACT

An implant delivery device (10) comprises a barrel (20), a plunger (30) and an inner sleeve (130) which comprises material stress means (140). The plunger is adapted to fit into a central bore of the inner sleeve in a sliding relationship and the inner sleeve in turn is adapted to fit into the bore of the barrel also in a sliding relationship. An alternative embodiment of the device generally comprises a barrel (20) and plunger (30) and an implant (100) loaded within the barrel, and a column of liquid (110) located proximally behind the implant. A user depressing plunger exerts a compression force on liquid, which is transferred onto the implant and the implant moves distally along the barrel toward the distal end (50).

The present invention relates to the field of implants, especially implantable biomaterials, and in particular, to a delivery device adapted for improved handling, positioning and discharge of an implant into a desired implantation site.

Medical/biomedical implants are typically delivered into a defect site or target tissue using a delivery device which typically comprises a tubular barrel through which the implant is “injected”, for example by using a plunger. For example, WO 2007/065150 discloses a delivery device comprising a curved tubular outer shaft defining an internal bore, and having proximal and distal ends, and an inner shaft adapted to fit therein in a sliding arrangement. An implant is advanced through the bore of the device by depressing the inner shaft.

One of the problems faced by surgeons when implanting a biomaterial into a patient relates to delivery. The barrel of the delivery device is inserted through an incision and positioned at or near the defect site, typically a hole or cavity in one or more tissues, following which the implant is advanced through the barrel, discharged through the distal end of the device and positioned within the defect site in the patient. In order to discharge an implant out of known barrel and plunger type delivery devices a user applies force to the plunger moving it distally through the barrel, the plunger either being in contact with or eventually contacting the implant, which itself then moves through the barrel coincident with the movement of the plunger. In advancing the implant along the barrel and discharging it out of the device, the direct contact made between the implant and the plunger can create problems, in that implants can become compressed or deformed (when no compression or deformation is desired), or worse still, trapped between the barrel and plunger resulting in damage as the pressure is applied. This is particularly the case with fragile or soft, for example spongy implants, or during procedures where the defect site is hard to access, or where the implant is travelling through an e.g. curved or bent barrel. Delivery of an implant in an undamaged state is obviously desirable.

Another problem relating to delivery of medical/biomedical implants is that they are often delivered in a dry state, with the surgeon taking measures to ensure that the implant remains dry right up to the point of insertion into a defect site. The implant when in situ tends to soak up liquid within the defect site, thereby drawing e.g. blood away from the tissues surrounding the defect site. Depending on the tissue being repaired and other factors such as e.g. reperfusion-rate, this absorption of liquid by the implant from the surrounding tissue can be detrimental to the healing process.

It is an object of the present invention to address the above stated problems by providing improved delivery devices that do not damage or provide unwanted or inappropriate compression forces on the implant during (i) handling, (ii) positioning of the implant within the barrel and (iii) during the implantation procedure itself, and which can reduce friction applied to the surface of an implant as it travels down a barrel further limiting damage to an implant. It is a further object of the invention to provide a device which can handle and deliver an implant in a wet state to prevent dehydration of the surrounding tissue upon implantation. It is a yet further object of the present invention to provide improved delivery methods.

According to a first aspect of the present invention, there is provided a delivery device suitable for implanting an implant into a cavity or hole comprising:
(i) a barrel defining a bore and having a proximal end and a distal end, and
(ii) a plunger within the bore and being in slidable relationship with the barrel, characterised in that the delivery device further comprises:
(iii) implant contacting means;
   wherein, in use the implant contacting means is adapted to limit longitudinal deformation of an implant as it travels through the bore by eliminating, or reducing the duration of, direct contact between the plunger and the implant.

The barrel defines proximal and distal ends with a bore disposed in the barrel, the bore extending longitudinally from the proximal end to the distal end and having a longitudinal axis.

The barrel of the delivery device may be tubular. The distal end of the barrel may terminate in or may comprise a distal tip. The device differs from traditional barrel and plunger arrangements by incorporating implant contacting means, essentially means to transfer force applied to the compression actuator or plunger to an implant, whereby contact between the compression actuator or plunger and the implant—which is often detrimental to the implant—is either reduced or preferably eliminated altogether.

By “distal end”, is meant the end furthest from a user, i.e. the end which is closest to the cavity, hole or implantation site and which may be inserted through a hole or an incision for implant delivery.

The terms “cavity” or “hole” as used herein should be understood in the broadest sense possible. The term is intended to refer to any relatively well-defined space, void, opening or recess in a substrate material such as a hole in a plaster board, tree trunk, foodstuff, or a bone defect site. The terms are intended to encompass irregular regions defined by an area or portion of the substrate material, as well as relatively well-defined defects such as bore holes and the like. Thus the terms may include apertures having a similar or smaller cross-sectional area than the entrance or opening and also apertures having a larger cross-sectional area than the entrance or opening. It will be apparent to the skilled person that such cavities or holes may be filled with a displaceable material such as a liquid, for example that may be displaced from the cavity or hole by the delivery device and/or implant.

Whilst the delivery device is generally directed to medical uses, it will be apparent to one skilled in the art that such devices may be utilised more widely, for example in the fields of building and manufacture, food production, and tree surgery, or indeed any other technical field where it is desirable to deliver an implant, plug, or body of material, which may be compressible or deformable into a cavity, hole or opening.

There is provided a delivery device suitable for implanting an implant into a cavity or hole comprising:
(i) a barrel defining a bore and having a proximal end and a distal end, and
(ii) a compression actuator within the bore, characterised in that the delivery device further comprises:
(iii) implant contacting means;
   wherein, in use the implant contacting means is adapted to limit longitudinal deformation of an implant as it transfers a compression force from the compression actuator to the implant.

The delivery device may comprise implant discharging means from the device. The implant discharging means may be the compression actuator, which may comprise a rod, piston or plunger adapted to fit within the barrel in a sliding relationship. The implant discharging means may be a plunger adapted to fit within the barrel in a sliding relationship. The compression actuator may be in slidable relationship with the barrel.

The implant may be a medical implant.

Preferably the device is adapted to deliver compressible, deformable or expandable implants, where the implant is compressible, deformable or expandable in a radial and/or longitudinal direction. Suitable implants include, for example, “plugs” used in building and manufacture comprising materials such as wood, resins, foams, plastics, wax or plaster materials, as well as medical and biological implants including, for example, grafts, hair follicle grafts, bone and dental implants, soft tissue implants and fillers such as silicone, bioplastique, arteplast, artecoll, collagen, chitosan and fat, pharmaceutical implants such as solid dosage forms, oestradiol implants, anticancer formulations and the like. It will be apparent to one skilled in the art that this list is not intended to be limiting and a variety of other applications will be possible.

The present inventors have previously developed a new biomaterial and methods for making the biomaterial. The present inventors have surprisingly found that this biomaterial has advantageous elastic and viscoelastic properties, this being disclosed in GB0721158.4 (filed 29 Oct. 2007). The implant may therefore be or may comprise a substantially elastic or viscoelastic porous material, for example as disclosed in PCT/GB04/004550 (filed 28 Oct. 2004), PCT/US2005/033873 (filed 21 Sep. 2005), PCT/GB2006/000797 (filed 6 Mar. 2006), and PCT/GB2007/003046 (filed 11 Aug. 2007) and GB0721158.4 (filed 29 Oct. 2007, the disclosure of each being incorporated herein in their entirety. The implant material preferably defines a rest state where, in the absence of stress acting on the material, it is in a non-deformed state. Under the application of stress, the material may strain and may be elastically deformable between the rest state and a fully-deformed state. The material may be plastically deformable beyond the fully-deformed state.

The term “substantially elastic” as used herein means a material which is capable of returning to or toward its original length, shape, etc., after being subjected to stress, for example stretching, deformation, compression, or expansion, or combinations thereof. In the absence or removal of stress the biomaterial may fully return to its rest state, or one or more dimensions of the biomaterial may partially or fully return to the rest state.

The term “viscoelastic”, as used herein means a material that exhibits both viscous and elastic characteristics when undergoing deformation, i.e. where the relationship between stress and strain depends on time. When the stress acting on the material is removed, the material may return to the rest state either instantaneously, or over a period of time. Alternatively, the material may be anelastic.

In preferred embodiments the delivery device is for medical/surgical use and the terms “cavity” or “hole” refer to defect sites in patients such as a humans or animals, for example domestic pets such as dogs and cats, zoological or ‘exotic’ animals such as elephants or animals used in farming, commerce and sport such as camels, dogs or horses and the like.

The cavity or hole, defect site or the target tissue may be comprised within bone, cartilage, tendon, ligament, meniscus, periodontal tissue, dentine, enamel, intervertebral discs, annulus fibrosus, and nucleus pulposus, or combinations thereof, for example an osteochondral surface.

The implant delivered by the device may be porous or non-porous. The term “porous” refers to a substrate or material that comprises pores such as macropores and/or micropores, holes or voids. Such pores, holes or voids may render the substrate or material permeable. The use of the term ‘permeable’ throughout this application may include penetration of a liquid or substance into or through the implant. In certain embodiments it may also include solubility of a component of the implant such that a liquid or substance is able to penetrate an implant, for example, by dissolution.

Preferably the implant comprises a porous biomaterial.

The term “biomaterial” as used herein means a material that is biocompatible with a human or animal body. The biomaterial may be comprised within, or may be, an implant or tissue scaffold.

The delivery device may comprise means for compressing or deforming an implant, for example in a radial and/or longitudinal direction. The barrel may comprise a neck region. Any or all of the barrel, neck region, distal end, or distal tip may be adapted to apply a stress to an implant being discharged from the device, the stress causing deformation of the implant toward a partially or fully deformed state.

By “discharged” is meant egressed or expelled from the barrel out of the device.

The barrel, distal end, distal tip and/or neck region may be the means for compressing or deforming an implant.

The barrel may have a greater cross-sectional area at its proximal end than at its distal end, whereby in use passage of an implant along the barrel in a distal direction in response to a force on the implant causes deformation of the implant toward a partially or fully deformed state.

By “cross-sectional” it is meant a cross-section perpendicular to the longitudinal axis of the barrel, i.e. a transverse section. Where the barrel is circular in cross section, the distal tip and/or distal end may have a smaller diameter than the rest of the barrel, and the proximal end. Thus, the transition from the larger cross-sectional area of the barrel to the smaller cross-sectional area of the distal end or distal tip may be progressive or sudden.

The stress on the implant may be applied by a progressive or sudden decrease in the cross-sectional area of the barrel or bore. All of or at least a section of one or more of the barrel, neck region, distal end, or distal tip or at least a section of the barrel may be inwardly tapered. The inwardly tapering barrel, distal end, distal tip and/or neck region may be the means for compressing or deforming an implant.

The distal tip may have a smaller cross-sectional area than the bore of the barrel whereby in use passage of an implant through the distal tip in response to a force on the implant causes deformation of the implant as it is discharged from the device. The distal tip or at least a section of the distal tip may be inwardly tapered. The degree of inward taper of the distal tip may be the same as or different to the degree of inward taper of the barrel. In some embodiments the distal tip may be tapered, whereas the barrel may lack an inward taper. The aperture defined by the distal end and/or distal tip may be smaller than that required to allow an implant to be discharged from the device without applying a stress to the implant as it passes through the distal end and/or distal tip.

The distal tip may define an adjustable aperture.

The device is preferably adapted to be used with implants where the transverse cross-sectional area of the implant may be greater than the transverse cross-sectional area of the bore of the barrel or the aperture defined by the distal end or distal tip.

Radial compression/deformation of an implant made of deformable or compressible material may be achieved by axially leading the implant through a space such as the barrel, which tapers in the transport direction and whose smallest diameter is smaller than that of the implant to be compressed. For example, where the barrel tapers inwardly towards its distal end, the tapering walls of the barrel apply stress on an implant as it travels along the barrel before passing through the distal end and out of the device. Stress may also be applied to an implant by the distal tip, or neck region, when present.

The delivery device may comprise one or more longitudinal slits cut through the distal end of the barrel, creating leaves, which preferably taper radially inwardly (i.e. toward the centre of the bore), and which may apply stress to an implant positioned within the distal end of the barrel. The leaves or the inward taper of the leaves may be adjustable. The stress may result in a compression on the implant which may provide a greater degree of precision and control during the delivery stage, and which may ensure that the implant can fit into the defect site, and optionally expand thereafter to fill the available space within the defect site.

The cross-sectional area of the bore or barrel may be a regular or irregular shape, and may for example be circular, oval, elliptical, hexagonal, or octagonal. The cross-sectional area of the bore may be shaped to match the cross-sectional area of a cavity or hole, thereby allowing precise alignment of the bore with the defect and delivery of correctly shaped implant into the defect. Thus, in particular embodiments, the barrel may be rotationally symmetrical, with the order of rotational symmetry varying dependent on the cross-sectional shape of the barrel. It will be apparent to one skilled in the art that the barrel may also comprise or be an irregular size, shape or configuration, and which may therefore have rotational symmetry of order 1 (i.e. be non-symmetrical).

The delivery device may comprise at least one alignment marker for orientation of the device with respect to a cavity or hole.

In preferred embodiments, the distal tip of the barrel comprises markings allowing a user, such as a surgeon, to determine the correct orientation and/or alignment of the distal end of the device relative to/within the defect site both prior to, during and/or after delivery of an implant. The at least one alignment marker is preferably a visual marking or indicator and may, for example, be printed, etched or grooved into the material of the distal tip. Markings may comprise graduations and/or gradations of text, numbers or characters, lines, symbols, different colours and the like. Preferably the marking(s) enable a user to determine the correct vertical orientation of the device within and with respect to the defect site, preventing an implant from being delivered at an angle to the base or sides of the defect site. Thus, preferred markings may include solid or dashed lines around the circumference of the distal end of the device, for example forming an unbroken or broken ring or band. Preferably the at least one alignment marker is/are provided along the barrel at a distance from the distal tip roughly equivalent to the depth of the defect site or roughly equivalent to the depth of the defect site plus an additional distance equivalent to a depth or layer of tissue or skin overlaying, for example, bone around the defect site. In use, and when the device is correctly orientated within the defect site, the marking(s) will align horizontally with the top of the defect site or overlaying tissue such that the markings are substantially parallel to the base of the defect site. If the device is incorrectly positioned, the marking(s) will be clearly visible to a user to be at an angle to the top of the defect site or overlaying tissue when viewed from one or more sides. In particular embodiments, the at least one alignment marker may be visible throughout use of the device. In further embodiments the at least one alignment marker may be visible when the device is incorrectly positioned and not visible when the device is correctly located, or vice versa. Such visual cues can then be used by the user to alter and correct the positioning of the device within the defect site such that the implant can be correctly deployed.

The cross-sectional area of the bore or barrel may match or be substantially the same as the cross-sectional area of one or more drill tips which may be used to prepare the defect site. In a preferred embodiment, the cross-sectional area of the bore is circular, and the implant delivery device is used to deliver implants having a circular cross-sectional area.

Preferably, the cross-sectional area of the bore is large enough to allow an implant to be advanced through the bore and into a defect site, but not so large that the implant has room to change its orientation within the bore. This can be important when delivering implants comprising two or more layers (multi-phased implants), or gradient implants, for example as disclosed in the applicants international patent application PCT/US2005/033873.

Thus, in particular embodiments, the barrel, distal end, neck region, and/or distal tip are adapted to apply stress to an implant being discharged from the device, the stress causing deformation of the biomaterial toward a partially or fully deformed state, thereby reducing its cross-sectional area.

Alternatively, the distal end of the device may be configured or adapted to control the size of the implant immediately prior to or substantially simultaneous with discharge from the device. Thus, the size of the aperture defined by the distal end or distal tip may be adjustable. The distal end of the device may comprise one or more leaves or other means which enable a user to adjust the size of the aperture or opening at the distal end. The distal end of the device may be adjustable between first and second positions wherein the opening defined by the distal end in the first position allows an implant to be discharged from the device with a larger size or cross-sectional area than is possible when in the second position.

Where the implant comprises a substantially elastic or viscoelastic material, the cross-sectional area of the distal end and/or distal tip may be smaller than the cross-sectional area of the bore of the barrel. The circumference and/or diameter of the distal end and/or distal tip may be smaller than the equivalent circumference and/or diameter of the bore of the barrel.

An elastic or viscoelastic material will return to or toward the rest state when the stress is removed. Therefore, a substantially elastic or viscoelastic material passing through the device may be deformed as it passes through the distal end and/or the distal tip; however, the material returns to or toward its original shape when it has been discharged from the device, i.e. when the stress applied by the various components of the device has been lessened or removed. Where the return to the rest state is not instantaneous, the implant can be positioned in a desired position within a defect site, and thereafter expand to or toward the rest state and completely fill the available space within the defect site. This then ensures that an optimal fit is provided between the implant and the defect site.

The compression actuator may comprise a pump or other means of applying a force either directly or indirectly to the implant in the barrel sufficient to effect movement and discharge of the implant from the device. Thus, a force may be applied to the implant in the barrel by a user actuating the compression actuator. Preferably the force is exerted longitudinally and acts to urge the implant along the barrel towards the distal end of the device. Preferably the applied longitudinal force is maintained until the implant is discharged from the distal end of the device. The compression actuator may itself be hollow, and may further comprise a bore or lumen, which may run along a longitudinal axis of the actuator. It will be apparent that the compression actuator may itself exert a compression force on an implant in a substantially longitudinal direction. The implant discharging means may be the same or different to the means for compressing an implant.

The delivery device may be adapted to contain a liquid within the barrel. The delivery device may comprise a liquid contained within the barrel.

The present inventors have surprisingly discovered that a compression force applied to the plunger or compression actuator may be indirectly applied to an implant through a liquid in the barrel of the delivery device. The compression force acting on the liquid also acts on an implant immersed within the liquid and which moves within the liquid in the direction of flow. Utilising a liquid in this manner has the advantage that the implant cannot become trapped between the wall of the barrel and the compression actuator. In addition the liquid assists in reducing friction between an implant and the wall of the barrel, reducing stress and/or damage as the implant travels along the barrel towards the distal end.

The implant contacting means may be a liquid, or cushion of liquid. The liquid or cushion of liquid may eliminate or reduce the duration of direct contact between the plunger and an implant, thereby limiting longitudinal deformation of the implant as it travels through the bore. By “cushion” it is meant a volume of liquid sufficient to transmit the force from the compression actuator or plunger to the implant, whilst eliminating or reducing the duration of direct contact between the compression actuator or plunger and the implant.

Thus, in a further embodiment of the first aspect of the invention, there is provided a delivery device that utilises a liquid to move and discharge an implant from the distal end of the barrel of the device.

The use of liquid to deliver an implant from an implant delivery device provides at least two advantageous functions. Firstly, it facilitates compatibility between the implant and the cavity or hole, for example by allowing a pre-soaked porous biomaterial to be delivered to a defect site in a physiologically compatible liquid. Secondly, it ensures that the implant—which may be fragile, spongy or prone to damage by virtue of it being wet—is protected by the liquid in the barrel as the implant is discharged from the device. This is particularly advantageous for porous biomaterials.

Upon application of a compression force the implant may move toward the distal end before being discharged through the distal end of the barrel. The amount of liquid discharging from the distal end upon application of the compression force may exceed the amount of liquid discharging from the distal end in the absence of the compression force.

The implant may be under stress and may be in a fully or partially-deformed state. Deforming the implant toward its fully-deformed state and keeping it in this state whilst in the barrel of the device can serve an important function, particularly if the material of the implant is highly porous because of a large pore size and/or large pore numbers. In the partially or fully deformed state, the pores within the material of the implant will be compressed, and this may result in an increased resistance to fluid passing through the implant material, thereby reducing the volume of liquid “flushing through” the implant. The compression of the pores and the resulting increased resistance to liquid flushing through thereby facilitates the discharge/movement of the implant from/within the barrel because a greater proportion of the liquid under pressure cannot bypass the implant (by e.g. flushing through) and instead exerts increased pressure on the implant, causing movement, relative to the same implant in its rest (i.e. non-deformed) state.

The implant is preferably immersed within the liquid contained within the barrel. The liquid may be added into the barrel to top up the amount, ideally in a volume such that the implant is fully immersed within the liquid when the barrel is orientated in a vertical position, and preferably in a horizontal position. Depending on the pore size of the implant material and the viscosity of the liquid being used, the liquid may be positioned only behind the implant within the barrel, i.e. not between the distal end of the implant and the distal tip of the device.

The device may further comprise liquid retention means to retain liquid within the barrel. The liquid retention means may comprise a valve, barrier, plug, stopper, sheath, tap, or any other means which can be used to retain liquid in the barrel by preventing it discharging through the distal end. The liquid retention means may comprise a barrier which prevents liquid from discharging from the distal end of the barrel. The liquid retention means may be positioned at, on, or within the distal end and/or distal tip. Alternatively, the liquid retention means may be positioned along the barrel or at the proximal end of the device.

The liquid may be retained due to surface tension at the distal end of the barrel, particularly where the bore at the distal end or distal tip is narrow (<5 mm).

The liquid may be retained by use of a barrier to prevent liquid from discharging from the barrel, and/or tilting the distal end of the barrel above a horizontal position. Where the proximal end is sealed, for example when a liquid compression actuator such as a plunger rod is positioned within the barrel, liquid can be prevented from discharging from the device by positioning the device so that the distal end or distal tip lies above a horizontal position, for example at or toward a vertical position.

The inventors have also surprisingly discovered that the liquid in the barrel does not wash through the implant and flush out any liquid absorbed within the implant. The liquid absorbed within the implant may substantially remain in the implant as the implant moves within the barrel. This surprising finding means that it is possible to pre-soak an implant in a first liquid, and immerse it in a second different liquid (which may be the same or different), whereby the first liquid substantially remains in the implant during any manipulation, positioning and discharging procedures.

Whilst the implant may comprise porous biomaterial, the porosity of the biomaterial may be insufficient to allow the liquid to be flushed through an implant in the barrel, particularly if the flow rate is high (e.g. >0.1 ml/second).

The liquid may protect the implant, or may act as a protective cushion to prevent or minimise damage occurring as the implant is contained or moved within the barrel. The use of a liquid to position an implant utilises hydraulic forces and relies on the incompressibility of the liquid within which the implant is immersed. The liquid may be incompressible or substantially incompressible. In contrast to known hydraulic devices, which utilise an incompressible column of liquid to perform work, the implant is in contact with the hydraulic fluid and is not remote from it. The implant contacting means may thus be a hydraulic fluid.

In the absence of liquid retention means, liquid contained within the device may discharge through the distal end of the barrel. The flow rate of the discharging liquid will be dependent on the viscosity of the liquid and the size of the opening defined by the distal end or distal tip. Where a small flow rate is desired, the opening may be adapted to restrict the discharge to small volumes, for example one drop at a time. A user placing their finger over and covering the distal end of the device may prevent liquid discharging from the device.

In the presence or absence of liquid retention means, the device may comprise liquid addition means adapted to add liquid into the barrel. The liquid addition means may be a cannula, tube or other delivery vessel which may be attached or attachable at one or more locations to the proximal end, the barrel, and/or the distal end. The liquid addition means may be positioned solely along the barrel, or at one end. The liquid addition means may positioned within a bore or lumen of a compression actuator or implant discharging means for example within a plunger rod adapted to fit the barrel of the device in a sliding relationship. The liquid addition means may be connectable to and/or connected a pump. The liquid addition means may convey liquid from a liquid source into the barrel of the device.

The liquid addition means, for example in the form of a tube connected to the proximal end, provides a means by which a user can add liquid into the barrel. The flow rate of addition may be adjustable, in the same way that the liquid retention means may be adjustable.

In a simple embodiment, the device may lack both liquid retention means and liquid addition means, and sufficient liquid may be maintained in the barrel by e.g. a user adding liquid into the barrel. By “sufficient liquid” it is meant enough liquid to contact an implant positioned in the barrel, preferably enough to immerse an implant, and more preferably, enough to fully immerse an implant.

To allow liquid to discharge from the barrel, a user may remove or disengage any liquid retention means which is engaged to prevent liquid from discharging from the barrel. Where the liquid retention means is a cover or barrier over the distal end or distal tip, a user may remove the barrier or cover. Where the liquid retention means is an e.g. tap, or valve, a user may manipulate or actuate the tap or valve to allow liquid to discharge. Alternatively, if the device is tilted beyond the horizontal to prevent liquid from discharging, a user may need to take no action where the compression force applied overcomes the forces of gravity keeping the liquid in the barrel.

An implant may be positioned or moved proximally within the barrel by applying a compression force to the liquid. The inventors have found that an implant contained within a cushion of liquid moves in the direction of flow of the liquid within the barrel, therefore to move an implant proximally within the barrel, liquid may have to discharge from the proximal end of the barrel, and/or liquid may be drawn up through the distal end.

The above-described process may thus allow a user to position an implant close to the distal end or distal tip, which may be desirable to obtain optimum precision and control when discharging an implant from the device.

Other embodiments of the device use different mechanisms to reduce or eliminate contact between the implant and the plunger or compression actuator.

In one embodiment, the implant contacting means may be adapted to contact an implant or hold an implant. The implant contacting means may comprise a compression ring, band or collet.

The implant contacting means may comprise at least two arms, the arms being adapted to be urged inwardly toward the longitudinal axis of the bore of the barrel. As the implant contacting means moves within the barrel, the arms may be urged inwardly through e.g. an inward taper of the barrel, neck region, distal end or distal tip.

The implant contacting means may thus be adapted to cause radial deformation of an implant. The radial deformation may be applied to the implant upon actuation of the device by a user. The radial deformation may be achieved by exerting at least one force on at least one side or edge of the implant. The radial deformation may be effected as the implant contacting means moves within the barrel, for example as a result of an inward taper of the barrel, neck region, distal end or distal tip.

The forces may be exerted on the implant at two or more regions around the perimeter of the implant. The forces may be exerted on the implant at two or more substantially equally spaced regions around the perimeter of the implant. The force or forces may be exerted on the implant along substantially the whole length of the implant. The forces may be applied evenly or unevenly around the perimeter of the implant. The forces may be the same or different strengths.

In preferred embodiments the implant contacting means is adapted to apply equal forces at regular intervals around the perimeter of the implant. The implant contacting means may comprise two, three, four, five, six, seven or eight arms, each being spaced at regular intervals and being adapted to be urged inwardly toward the longitudinal axis of the bore of the barrel. As the arms are urged inwardly, substantially equal radial deformation forces are applied evenly to the implant.

The device may further comprise:
(iv) an inner sleeve having a proximal end and a distal end, wherein the distal end is operably linked to the implant contacting means, the inner sleeve being in slidable relationship with both the barrel and the plunger,
   wherein in use the inner sleeve applies a force that is transferred to an implant to move the implant along the barrel in a distal direction.

In use an implant may be positioned distally from the plunger and wherein the inner sleeve is adapted to move the implant to a first position towards the distal end of the barrel and the plunger is adapted to move to a second position at the distal end of the barrel whereby movement of the plunger toward the second position pushes the implant out of the device.

The plunger may be adapted to move together with the inner sleeve to the first position in the barrel.

The device may further comprise actuation means for initiating and/or assisting the discharge of an implant from the distal end of the barrel, the actuation means being in operative communication with the plunger and/or inner sleeve.

The actuation means may be adapted to move the plunger and the inner sleeve to a first position and subsequently to move the plunger to a second position whereby in use movement to the first position causes deformation of an implant and movement toward the second position pushes the deformed implant out of the device.

The actuation means may comprise first and second actuation means wherein, the first actuation means is adapted to move the second actuation means, plunger and where present inner sleeve to a first position and the second actuation means is adapted to move the plunger to a second position whereby in use, movement to the first position causes deformation of an implant and the movement toward the second position pushes the deformed implant out of the device.

The device may further comprise first and second actuation means for initiating and/or assisting the discharge of an implant from the distal end of the barrel, the first actuation means being adapted to move the second actuation means and inner sleeve to a first position and the second actuation means being adapted to move the plunger to a second position whereby in use, movement to the first position causes deformation of an implant and the movement toward the second position pushes the deformed implant out of the device.

When present, the inner sleeve may be hollow comprising a bore or lumen. In particular embodiments, the implant discharging means may be a rod, piston or plunger adapted to fit within a bore or lumen of an inner sleeve in barrel in a sliding relationship. Hence, when the device comprises both an inner sleeve and a implant discharging means, it is preferred that the implant discharging means slidably engages the inner sleeve, extending into and/or through the bore or lumen of the inner sleeve.

The delivery device may comprise a handle. The delivery device may comprise a housing, which may define a handle. The implant discharging means, barrel and/or inner sleeve may comprise a handle, gripping means, or one or more sections to facilitate actuation, for example by the application of pressure by a user.

The handle may partially cover the barrel of the device at its proximal end and leaving at least the distal end of the barrel exposed. The handle may be between 70-95 mm in length. The handle may be adapted or shaped such that when the handle is gripped by a user, the delivery device is held in a particular orientation. The handle and/or barrel may comprise one or more flattened sections adapted to engage with the fingers of a user. The barrel may comprise two such sections, adapted to engage with the first and second fingers, or the second and third fingers of a user.

The implant discharging means and/or inner sleeve may also comprise one or more sections or parts adapted to enable actuation, for example by engaging with the thumb or a finger of a user. Thus, a compression force may be applied to an implant in the barrel by a user holding the barrel with their fingers, and depressing the implant discharging means and/or inner sleeve with their thumb, analogous to the depression of a plunger in a syringe barrel. In particular embodiments the inner sleeve and compression actuator are operated sequentially. For example a user may first apply a radial compression force to an implant by actuating or depressing the inner sleeve, subsequently a longitudinal compression force sufficient to discharge an implant from the device is applied to the implant by actuating or depressing the implant discharging means. In particular embodiments, the implant discharging means cannot be actuated until at least a first actuation of the inner sleeve has occurred. Preferably the device comprises at least first and second actuation means being in operative communication with the inner sleeve and/or the implant discharging means.

Preferably the delivery device further comprises biasing means that cooperate with the implant discharging means. On actuation of the implant discharging means, the biasing means act to displace at least a part of the implant discharging means or plunger within the barrel, for example, in the event that the implant meets resistance as it is discharged from the distal end of the barrel. For example, if the distal end of the barrel is blocked or obstructed, such as by the base of a hole or cavity, the biasing means enables the implant discharging means to complete its full actuation movement. In doing so this in turn causes the biasing means to, for example, compress, compensating for the reduced range of travel whilst maintaining a longitudinal force on the implant. The longitudinal force will be maintained until such time as the blockage or obstruction is removed, at which point the biasing means may expand such that the implant is discharged from the distal end of the device. In particular embodiments the implant discharging means comprises two or more parts. The biasing means may be, by way of non-limiting example, a spring or elastomer, integral sprung section or living hinge although other suitable alternatives will be apparent to the skilled person. In particular embodiments, the implant discharging means comprises a rod or plunger in operative communication with actuation means. In this embodiment, the biasing means is operatively positioned between the rod or plunger and actuation means.

Hence, when the distal end of the delivery device is placed at the base of a cavity or hole, the biasing means ensure that the implant will be released at substantially the same time as the delivery device is withdrawn from the cavity or hole.

The means for compressing an implant may comprise material stress means that can be adjusted, either manually or automatically, to exert stress on the implant and deform it toward a fully or partially-deformed state, for example by contracting inwardly, more particularly radially inwardly. The stress may be applied in either a uniform way, or in a way that allows localised stress to be exerted on at least a part of the implant, thereby promoting movement toward the fully or partially-deformed state. Preferably radial compression is achieved by exerting a substantially equilateral circumferential force but equally the force may be an even or uneven force(s) applied to the implant, preferably at substantially equally spaced regions around the circumference or edge of the implant. The material stress means may comprise a radial collar, or other means, for example, means by which the barrel can be locked in a chosen bore size, thereby exerting stress on the implant, if desired. In alternative embodiments, the material stress means may comprise a compression ring or one or more ‘arms’ extending from an end of an inner sleeve within the bore, the inner sleeve having an axis and proximal and distal ends and extending along the axis of the barrel. Where present, preferably the one or more arms extend substantially longitudinally from the distal end of the inner sleeve. The arms are separated by a gap enabling them to be urged towards one another, effectively reducing their cross-sectional dimension. In embodiments when a single arm is used, the arm will be in the form of a C-shape, for example and may have regions that can overlap when acted on by compression force(s). Radial deformation or compression of an implant made of compressible material, may be achieved by axially leading the material stress means through a space such as the barrel, which tapers in the transport direction and whose smallest diameter is smaller than that of the body to be compressed. For example, the barrel may taper inwardly towards its distal end. The taper may be adapted to apply stress on the material stress means as it travels along the barrel which in turn applies, or transfers, a force to the implant. Thus, in particular embodiments, the barrel, distal end and/or distal tip are adapted to apply stress to a material stress means which in turn applies or transfers the stress to an implant being discharged from the device, the stress causing deformation of the implant toward a partially or fully deformed state as it travels towards the distal end of the barrel. In other embodiments, the barrel of the device may be adjustable for a variable bore size. For example, the barrel may be twisted to adjust the bore size, analogous to rolling a sheet of paper into a tube. In particular embodiments, the barrel may be rotationally symmetrical but it will be apparent to one skilled in the art that the barrel may also comprise or be an irregular size, shape or configuration, for example non-symmetrical. Utilising a compression ring or one or more arms has the advantage that the implant cannot become trapped between the wall of the barrel and the compression actuator. In addition the possibility of an implant being damaged due to friction as it travels along the barrel towards the distal end is reduced because it is the compression ring or one or more arms that encounter the greatest amount of friction with the barrel. The implant, in contrast, is generally not moving within the compression ring or arms and therefore only encounters minimal friction as it is discharged from the device.

In particular embodiments, the implant is in contact with the liquid. The implant may be immersed within the liquid, either partially or fully. Preferably, the implant is fully immersed within the liquid. The volume of liquid required for full immersion will depend on the orientation of the barrel, where a horizontal orientation will require more liquid than if the barrel is orientated vertically. By fully immersed it is meant fully immersed when the barrel is in an at least vertical position, and preferably in a horizontal position also. The barrel may therefore be partially or fully filled with liquid. The liquid may be substantially contained “behind” the implant, i.e. between the proximal end of the implant and the proximal end of the barrel.

For porous implants, liquid may be absorbed within the implant. The liquid absorbed in the implant may be the same as the liquid in the barrel, or the liquids may be different.

The delivery device may comprise a medical implant for implantation into a cavity or hole.

Rather than implant a dry material into a cavity or hole, it may be desirable to prepare the implant before implantation by contacting it with a liquid either prior to loading into the delivery device, or after loading into the delivery device. The porosity of, for example, biomaterials used in known implants means that the material may absorb any liquid with which it is contacted.

According to a second aspect of the present invention there is provided a process for preparing a medical implant prior to implantation in a patient the process comprising:

with a delivery device according to a first aspect of the present invention:
(i) contacting a medical implant with a liquid and loading the implant into the barrel, or
(ii) loading a medical implant into the barrel and contacting the implant with a liquid.

According to other aspects of the present invention, there is provided a method for preparing a porous biomedical implant prior to implantation in a patient, the process comprising:
(i) contacting a porous biomedical implant with a liquid and loading the implant into the barrel of a delivery device, or
(ii) loading a porous biomedical implant into the barrel of a delivery device and contacting the implant with a liquid.

The implant may be immersed within liquid contained within the barrel.

The implant can be pre-soaked and delivered containing the liquid which will be found at, in, or around the cavity or hole. This pre-soaking and delivery of an implant in a wet state can solve the problem relating to dehydration of the surfaces surrounding the cavity or hole by an otherwise dry implant. Thus, pre-soaking of an implant may prevent cracking or shrinking of the surfaces surrounding a cavity or hole through dehydration. In other embodiments, the implant may be pre-soaked in an adhesive or component of a two-part adhesive to facilitate bonding of the implant to the surfaces of the cavity or hole.

When the implant is a biomaterial, pre-soaking the implant may promote or facilitate healing and minimise trauma to a cavity, hole or defect site after implantation. The biomaterial may be soaked or contacted with a liquid that is physiologically compatible, for example blood, serum, plasma, CSF, bone marrow supernatant and that may contain one or more biomolecules selected from the group consisting of: cytokines, growth factors, hormones or a combination thereof, and/or one or more pharmacological agents such as antibiotics, anti-clotting agents and the like.

When the implant is a biomedical implant it may comprise a substantially elastic or viscoelastic porous biomaterial that is deformed toward a partially or fully deformed state either before or after contacting with the liquid or before or after loading the implant into the barrel of a delivery device.

The methods may comprise maintaining the biomaterial in its partially or fully deformed state. The porous biomaterial may act like a wick and gently draw up the liquid. The amount of liquid absorbed by different biomaterials will vary, and will depend inter alia on the respective chemical and physical properties. Depending on the liquid used, the resulting liquid containing implant may be directly compatible with liquid encountered at or in the implantation site. Thus, if the defect site comprises blood, it may be desirable to pre-soak an implant prior to delivery in blood, serum or plasma, which may be derived from the patient. The same principle applies for CSF, bone marrow supernatant, and indeed any other liquid which might be encountered at a defect site either before, during or after the implantation procedure.

The methods may comprise allowing liquid to absorb into the biomedical implant. The amount of time required for absorption may vary, and will depend on the liquid being absorbed and the porosity of the implant. The absorption time may be between 1 second to 24 hours, where less viscous liquids such as saline will absorb more quickly than more viscous liquids such as blood. The absorption time may be between 2 seconds and 12 hours, 5 seconds and 1 hour, 10 seconds and 10 minutes, or 20 seconds and 5 minutes.

The methods may comprise adding a liquid into the barrel prior to loading of the implant, and/or after loading of the implant.

The methods may comprise adding a second liquid into the barrel prior to loading of the implant, and/or after loading of the implant. The liquid and second liquid may be different, and the process may comprise mixing the liquids together. For example, a user may take an implant pre-soaked in one liquid, and load the implant into the device. By adding a second liquid, the user may wish to mix the liquid in the implant with the second liquid. This can be done by applying a compression force to the liquid, causing it to flush through the biomaterial, thereby mixing the liquids. Depending on the pore size of the biomaterial, this mixing step may require several flushes because in some embodiments the biomaterial is surprisingly resistant to being flushed through, particularly if it is in a fully or partially deformed state, where the pore size can be small causing an increased resistance to liquid passing through it. Thus, if the biomaterial is not deliberately flushed through, it is possible to substantially preserve the liquid in the biomaterial and keep it separate from the second liquid which may be contained within the barrel of the device. Alternatively, if a user desires mixing, a deliberate and vigorous flushing may be required to fully mix the liquid in the biomaterial and that contained in the barrel. Any mixing step may also be effected by inverting the device one or more times.

The methods may further comprise preventing liquid from discharging from the barrel. The process may comprise retaining sufficient liquid within the barrel to keep the implant immersed within liquid. Liquid may be retained in the barrel due to surface tension at the distal end of the barrel. Liquid may be retained by use of a barrier to prevent liquid from discharging from the barrel, and/or tilting the distal end of the barrel above a horizontal position.

After the implant is loaded into the barrel, and when the implant is contacting liquid, the process may further comprise positioning the implant distally within the barrel, comprising:
(iii) allowing liquid to discharge from the barrel;
(iv) applying a compression force to the liquid, the compression force discharging liquid though the distal end of the barrel, wherein the implant is simultaneously moved distally within the barrel, and
(v) removing the compression force when the implant has reached the desired position within the barrel.

The methods may further comprise retaining sufficient liquid within the barrel to keep the implant immersed within liquid after the implant has reached the desired position within the barrel. The process may further comprise adding sufficient liquid into the barrel to keep the implant immersed within liquid after the implant has reached the desired position within the barrel.

The methods may comprise using a barrier to prevent liquid from discharging from the barrel.

In further embodiments of the present invention, there is provided methods for discharging a biomedical implant from a barrel of a delivery device, the process comprising:
(i) taking a delivery device comprising a barrel having a distal end through which an implant can be discharged, the barrel containing a porous biomedical implant immersed within liquid, and optionally comprising a barrier to retain liquid within the barrel;
(ii) removing any barrier, and
(iii) applying a compression force to the liquid, the compression force discharging the liquid though the distal end of the barrel, wherein the implant is simultaneously discharged through the distal end of the barrel with the liquid.

According to a third aspect of the present invention there is provided a process for discharging a medical implant from a barrel of a delivery device according to a first aspect of the present invention, the process comprising:
(i) removing any barrier, and
(ii) applying a compression force to the liquid, the compression force discharging the liquid though the distal end of the barrel, wherein the implant is simultaneously discharged through the distal end of the barrel with the liquid.

The compression force may be applied using a compression actuator which may be a plunger. The liquid may be implant contacting means. The liquid preferably eliminates or reduces the duration of contact between the compression actuator or plunger and the implant.

In a third aspect, there is further provided a process for discharging a medical implant from a barrel of a delivery device according to a first aspect of the present invention, the process comprising:
(i) applying a force to an implant to move the implant along the barrel in a distal direction wherein the movement causes deformation of the implant and
(ii) applying further force to the implant to move the implant further along the barrel in a distal direction and discharge the deformed implant out of the barrel of the device.

The force and further force may be applied in separate sequential steps, or in a single step.

In response to a user actuating the implant contacting means, for example by actuating the compression actuator or plunger, the implant contacting means may apply a force to the implant to move the implant along the barrel in a distal direction.

There is provided a process for discharging a medical implant from a barrel of a delivery device according to a first aspect of the present invention, the process comprising:
(i) a user actuating the implant contacting means to apply a force to an implant to move the implant along the barrel in a distal direction, and
(ii) applying further force to the implant to move the implant further along the barrel in a distal direction and discharge the deformed implant out of the barrel of the device.

The implant contacting means may cause radial deformation of the implant. The movement of the implant within the barrel may cause deformation of the implant.

Where the implant contacting means comprises two or more arms, movement of the implant along the barrel in a distal direction in response to the force may cause the at least two arms to be urged inwardly toward the longitudinal axis of the bore of the barrel.

The arms of the implant contacting means may hold or grasp the implant. Alternatively, the arms of the implant contacting means may be urged inwardly, contacting the proximal end of the implant, and pushing the implant through the barrel in a distal direction. Although the arms can push the implant in a distal direction, they are preferably adapted to grasp the implant and in doing so provide greater control of the implant and its deformation as it passes through the barrel and out of the device. In a preferred embodiment, the arms are urged inwardly upon actuation of the device, the arms evenly radially deforming the implant, thereby allowing precise delivery of a wet, spongy and/or fragile implant in a partially or fully deformed state.

In response to a user actuating the plunger, the plunger may apply the further force to move the implant further along the barrel in a distal direction and discharge the deformed implant out of the barrel of the device.

In response to a user actuating the implant contacting means, the arms of the implant contacting means may apply the further force to move the implant further along the barrel in a distal direction and discharge the deformed implant out of the barrel of the device.

There is provided a method of implanting an implant in a cavity or hole comprising:
(a) loading an elastic or viscoelastic implant into the barrel of a delivery device, said implant being deformable between a rest state and a partially or fully deformed state,
(b) applying a deformation force to the implant causing it to partially or fully deform, and
(c) discharging the deformed implant from the barrel of the delivery device into the cavity or hole,
   wherein upon discharge of the implant from the barrel of the device into the cavity or hole the implant returns toward its rest state.

According to a fourth aspect of the present invention there is provided a method of implanting a medical implant into a cavity or hole in a patient, the method comprising:
(i) providing access to the cavity or hole through a surgical incision in the body of the patient;
(ii) discharging an implant by the method of the third aspect of the present invention.

The process or method may further comprise allowing the implant to expand to or toward its non-deformed state after being discharged from the device.

There is provided a method of implanting a biomedical implant into a defect site in a patient, the method comprising:
(i) providing access to the defect site through a surgical incision in the body of a patient,
(ii) taking a delivery device comprising a barrel having a distal end through which an implant can be discharged, the barrel containing a biomedical implant and optionally a liquid,
(iii) applying a deformation force to the implant,
(iv) discharging the deformed implant from the barrel of the delivery device into the defect site.

According to still further aspects of the present invention, there is provided a method of implanting a biomedical implant into a defect site in a patient, the method comprising:
(i) providing access to the defect site through a surgical incision in the body of a patient;
(ii) taking a delivery device comprising a barrel having a distal end through which an implant can be discharged, the barrel containing a biomedical implant immersed within liquid, and optionally comprising a barrier to retain liquid within the barrel;
(iii) removing any barrier;
(iv) applying a compression force to the liquid, the compression force discharging the liquid though the distal end of the barrel, wherein the implant is simultaneously discharged through the distal end of the barrel with the liquid, into the defect site.

The methods may further comprise allowing the biomaterial to expand to or toward its non-deformed state. One or more dimensions of the biomaterial in its fully or partially-deformed state may be less than equivalent dimension/dimensions of the defect.

The methods may comprise attaching the delivery device to the defect site or a target tissue.

There is provided a method of implanting a biomedical implant into a defect site in a patient, the method comprising:
(i) providing access to a defect site through a surgical incision in the body of a patient;
(ii) inserting an obturator into the bore of a delivery device comprising a barrel having a distal end through which an implant can be discharged;
(iii) inserting the delivery device and obturator positioned therein at the defect site;
(iv) removing the obturator from the bore;
(v) inserting a biomedical implant and a volume of liquid into the barrel, the implant being fully immersed in the liquid;
(vi) applying a compression force to the liquid, the compression force discharging the liquid though the distal end of the barrel, wherein the implant is simultaneously discharged through the distal end of the barrel with the liquid, into the defect site.

The methods may further comprise using liquid retention means to retain liquid within the barrel until the compression force is applied. To allow liquid to discharge from the barrel, a user may remove or disengage any liquid retention means which is engaged to prevent liquid from discharging from the barrel. Where the liquid retention means is a cover or barrier over the distal end or distal tip, the methods may comprise removing the barrier or cover. Where the liquid retention means is an e.g. tap, or valve, the methods may comprise manipulating or actuating the tap or valve to allow liquid to discharge. Alternatively, if the device is tilted beyond the horizontal to prevent liquid from discharging, a user may need to take no action where the compression force applied overcomes the forces of gravity keeping the liquid in the barrel.

The methods may further comprise retaining sufficient liquid within the barrel prior to applying the compression force. The methods may comprise using a barrier to prevent liquid from discharging from the barrel. The process may further comprise adding sufficient liquid into the barrel to keep the implant immersed within liquid before or during the implantation procedure.

The methods may further comprise preparing the defect site with a surgical drill device prior to delivery of the implant into the defect site. Preparing the area may involve drilling the edges of the defect to modify its shape, improve its structural integrity, and create a better fit between implant and defect.

The methods may comprise applying a compression force to the liquid by actuating the liquid compression actuator.

The implant preferably does not contact the compression actuator. The implant may remain immersed in liquid throughout the discharging process.

The methods may further comprise implanting cells in the patient. Cells may be seeded on the biomaterial or may be added separately. The cells may be stem or progenitor cells.

The methods may further comprise administering one or more biomolecules selected from the group consisting of: cytokines, growth factors, hormones or a combination thereof, and/or one or more pharmacological agents. A preferred hormone is parathyroid hormone (PTH).

When the delivery device comprises a compression actuator, the compression force may applied by actuating the compression actuator. Where the compression actuator is a plunger rod or piston, actuation may be achieved by a user depressing the plunger rod or piston.

Upon application of the compression force the implant may move toward the distal end before being discharged through the distal end of the barrel. The amount of liquid discharging from the distal end upon application of the compression force may exceed the amount of liquid discharging from the distal end in the absence of the compression force.

The orientation of an implant within the barrel may be maintained as it passes through the barrel and through the distal end and/or distal tip. Maintaining orientation may be desirable where non-uniformly porous biomaterials, or composite or gradient biomaterials are comprised within the implant.

The each liquid may be a physiologically compatible liquid, which may be saline, buffered saline, or water, or an autologous or heterologous biological liquid selected from the group consisting of: blood, serum, plasma, CSF, and bone marrow supernatant. The liquid absorbed in the implant and the liquid in the barrel may be the same or different.

According to a fifth aspect of the present invention there is provided use of a delivery device according to a first aspect of the present invention for implanting a medical implant into a cavity or hole. In preferred embodiments the implant is a porous biomedical implant for implantation into a patient.

According to a sixth aspect of the present invention there is provided kit of parts comprising a delivery device according to a first aspect of the present invention and further comprising at least one of: at least one medical implant, at least one delivery device, at least one obturator, at least one surgical drill device, at least one surgical drill tip, and instructions for a user.

The kit may comprise two or more delivery devices, which may each differ in one or more dimensions, thereby allowing a user to select the delivery device which is most optimally shaped for the size of defect and the procedure at hand. The kit preferably comprises instructions for a user.

Preferably, the kit comprises a plurality of implants, which may each differ in one or more dimensions. In an alternative embodiment the implant may be shaped in an e.g. block, and may have to be cut or trimmed to the size of the defect by a person performing the procedure.

The implant delivery kit may further comprise an obturator, the obturator comprising a shaft having proximal and distal ends, the shaft being adapted to fit within the bore of the delivery device in a sliding relationship. The distal end of the shaft may comprise a rounded or bullet-nosed tip. The obturator is insertable into the bore of the delivery device. When positioned within the bore of the delivery device, the distal end of the shaft and/or the tip is adapted to protrude from the distal end of the delivery device, and the rounded or bullet-nosed tip is adapted to facilitate the advancement of the delivery device through the incision toward the site of a defect. The rounded or bullet-nosed tip is adapted to minimise any damage to the tissue within an incision. In one embodiment, the obturator and/or the rounded or bullet-nosed tip is coated with one or more of heparin, an antibiotic, or a lubricant. The proximal end of the obturator may comprise means to contact the stop or abutment of the delivery device.

The kit may further comprise a surgical drill device for preparing an area containing a defect within a target tissue, the surgical drill device comprising a drill shaft having proximal and distal ends and a drill tip, the drill shaft being adapted to fit within the bore of the delivery device in a sliding relationship, and to be rotatable about its longitudinal axis. The rotation of the drill tip may be performed manually or may be power assisted. The drill tip may have a cross-sectional area which is similar to that of the implant, so that when the defect site is prepared (i.e. drilled out), the resulting defect is essentially the same shape and size as the drill tip. The shaft and/or the drill tip may be graduated or otherwise marked to show the depth of insertion of the drill tip/shaft. The surgical drill device may further comprise an attachment tip on the proximal end of the drill shaft, the attachment tip being configured to be received in a rotation device. The surgical drill device may further comprise a bore extending longitudinally from the distal end to the proximal end, the bore being adapted to fit a guide wire in a sliding relationship.

The obturator, plunger rod and surgical drill device may be resilient or flexible, and may be shaped to slide within straight, hinged, and/or curved sections of the bore of the delivery device. The obturator, plunger rod and surgical drill device may comprise the same material as the delivery device. The obturator, plunger rod, and/or surgical drill device may each comprise a handle at their proximal end. The handle may comprise a plastics material, or may comprise the same material as the delivery device.

The kit may comprise a plurality of delivery devices, the devices having various dimensions; and/or a plurality of surgical drill bits having various drill dimensions; and/or a plurality of obturators having various obturator dimensions; and/or a plurality of plunger rods having various plunger dimensions; and/or a plurality of guide wires having various guide wire dimensions; and/or a plurality of implants having various implant dimensions.

The various tools of the kit may be fully or partially disposable. In one embodiment, it is envisaged that the kit is entirely disposable, and is used for delivering one or more implants into one or more defects within the same patient. Alternatively, one or more of the tools, or indeed parts of the tools within the kit may be re-useable, for example the surgical drill device, the drill tip, and/or the attachment. The materials of the various tools of the kit and the tools themselves may therefore be autoclavable and resistant to detergents, gamma radiation, ethanol, and indeed any other chemicals and processes required to clean and sterilise the tools in preparation for another surgical procedure.

Further Delivery Device Features

The barrel may be opaque, transparent, translucent, or combinations thereof. At least a part of the barrel may be transparent or translucent. Preferably at least the distal end of the barrel is transparent or translucent, and more preferably the entire barrel is transparent or translucent. This may allow a user to visualise an implant positioned within it. The barrel may be coloured or tinted. The barrel may comprise graduations or markings. The device may comprise a material compatible with surgery, i.e. a material which can be readily cleaned, sterilised, irradiated, or combinations thereof, for example medical grade stainless steel or plastics material.

The delivery device may comprise one or more windows within the distal end of the barrel for visualising an implant positioned within the bore. The window may extend partially or fully along the length of the barrel, and may extend from one or more points about the circumference of the barrel, either in a regular or irregular spacing, thereby allowing a user to easily visualise an implant positioned within the bore without having to rotate the delivery device within a surgical incision. The window may be positioned in correlation with the shape or positioning of the handle, such that when a user grips the handle of the delivery device and holds the device in the prescribed orientation, the window may be positioned to allow visualisation of an implant within the bore, without the need to rotate the device within the surgical incision.

The barrel of the delivery device may be straight, curved, or bent or may comprise one or more regions each of which may be straight, curved, or bent. The barrel may be curved or bent at or near its distal end. The barrel may contain one or more curves or bends of up to 180 degrees, more preferably 5-90 degrees, and more preferably still 10-80 degrees. The bend or curve may be abrupt or smooth.

The barrel may comprise a flexible section at or near the distal end of the barrel, with the flexible section allowing the distal end of the barrel to be bent or shaped into different angles and positions. The barrel may comprise at least one hinge at or near the distal end of the barrel. A curved or bent barrel, or a barrel comprising one or more flexible sections or hinges may facilitate access to a defect site and improve positioning of the delivery device at a defect site. The flexible section is preferably flexible enough so that the angle and orientation of the distal end can be adjusted by hand, but is able to retain its shape while the delivery device is being positioned over a defect.

The barrel may comprise a rigid material and/or a flexible material. The barrel may be deformable to allow a user to adapt the shape to match the cross-sectional area of the defect. The rigid material may comprise stainless steel which is preferably surgical grade stainless steel.

The handle, barrel and/or other components of the device may comprise a material selected from the group consisting of: nylon, polytetrafluoroethylene (PTFE), acrylonitrile butadiene styrene (ABS), polycarbonate, polymethylmethacrylate, glass, carbon fibre, and latex. The handle and/or barrel preferably comprises a surgically compatible plastics material, or any other plastics material that is non-toxic, autoclavable, gamma resistant and/or ethanol compatible. The flexible material may comprise a rubber material and/or a plastics material, or any surgically compatible material which provides the required flexibility, but which is able to maintain its shape throughout the surgical procedure.

The barrel may be coated or plated with one or more surgically compatible materials which may include one or more of heparin, antibiotics, or a lubricant.

Preferably the delivery device is sized for use in arthroscopic surgical procedures. The delivery device may be between 120-155 mm in length.

The delivery device may be fully or partially disposable. The barrel and/or handle may be re-useable. The materials of the delivery device may be autoclavable and resistant to detergents and ethanol and the like. Preferably the delivery device is easily dismantled into its separate component parts, for example, facilitating sterilisation or replacement of particular components.

The device allows for the delivery of implants, where the implants may have a range of diameters for example between 2 and 20 mm. For arthroscopic use, the diameter of implants may be 15 mm or less. The implants may be single or multi-layered, for example as disclosed in PCT/GB04/004550, PCT/US2005/033873, PCT/GB2006/000797, PCT/GB2007/003046, and GB0721158.4.

In some embodiments the barrel may comprise a stop defined at or toward its proximal end, the stop being configured to provide an abutment for limiting the length of travel for other surgical instruments adapted to fit within the bore of the delivery device. In one embodiment the stop is a step formed by an increase in the diameter of the bore at or toward the proximal end of the barrel. The stop may engage with a corresponding stop on the shaft of other surgical instruments, thereby limiting their travel within the bore of the delivery device. The stop can therefore be useful in e.g. preventing a surgical drill device from drilling too far into the tissue of a patient.

The barrel may comprise a threaded outer surface to facilitate insertion of the delivery device into an incision. In this way, a user of the delivery device may facilitate insertion of the device into an incision by rotating the device one way and rotating the device in the opposite direction to facilitate its removal from the incision.

In particular embodiments the barrel may also comprise an attachment for securing the distal end to a defect or an area containing the defect. The attachment may extend from the distal end of the delivery device. The attachment may comprise one or more projections extending from the distal end of the barrel, each projection defining a proximal and distal end and being adapted to penetrate for example, the base of a cavity or hole, such as the tissue of a patient and thereby facilitate anchoring of the delivery device in position at a defect site.

Each projection may comprise a prong, tooth, extension, spike, or spur. Each projection may be adapted to attach the delivery device in position over the defect, preferably so that the bore is aligned as accurately as possible with the defect.

Each projection may comprise a blade at its distal end. One or more of the projections may be configured to incise about an area containing the defect. The blade is preferably adapted to be inserted into a defect or around the site of a defect. The blades may be adapted so that a rotation of the delivery device about its longitudinal axis promotes insertion of the blade into a tissue of the patient.

One or more projections may be graduated to facilitate assessment of depth penetration in the target tissue and facilitate correct orientation of the delivery device, which is optimally 90 degrees relative to the e.g. bone surface. The one or more projections may be coloured, or shaped in such a way as to allow a user to gauge the depth of insertion/penetration. In a preferred embodiment a series of indentations are marked on the projection, preferably comprising a deeper, longer or otherwise larger indentation to mark every other millimetre, or every 5 mm, or other desired depth increment.

The projections may extend 1-20 mm beyond the distal end of the barrel, and more preferably 2-10 mm beyond the distal end of the barrel. The projections may each extend the same distance from the distal end of the delivery device, or they may extend different distances. The distance of extension of each projection from the barrel may be adjustable.

The delivery device may comprise three projections, for example in a trident configuration. Each projection may be spaced equally or regularly around the circumference of the barrel. An equal or regular spacing may provide more stability when the delivery device is positioned over a defect.

The delivery device and/or the attachment may comprise a suction device, which may be adapted to attach to the defect, or the tissue surrounding the defect. The barrel may comprise a seal or sealing means at its proximal and/or distal end. The barrel and/or attachment may be adapted to create a seal around the defect, and the device may comprise pressure reducing means to allow the pressure in the bore to be reduced, thereby creating a negative pressure with respect to the atmosphere outside the bore, and thereby facilitating the pressure-mediated attachment of the delivery device to the defect. The pressure within the bore may be reduced using a pump, or by cooling the air within the bore, or heating the air outside of the bore. The distal end of the delivery device may be shaped to promote such pressure-mediated attachment. The distal end may comprise smooth and/or rounded edges which may promote the creation of a pressure seal around the defect. The delivery device preferably comprises means to equalise the pressure in the bore with the pressure outside the bore, thereby enabling a user to detach the delivery device from the patient without causing any damage to the tissue, or the freshly-repaired defect.

The distal end and/or the attachment may comprise one or more magnets adapted to magnetically attach to one or more magnets positioned in or around the defect. Alternatively, electrostatic forces may be used to attach the delivery device in position.

The attachment may comprise an adhesive or cement adapted to bond to the target tissue and/or the defect, or a tissue located near the defect, or a structure positioned in or about the defect. Any suitable surgical adhesive or cement is envisaged and these are well known to a person skilled in the art.

The attachment may comprise a dilation device, the dilation device being adapted to fit within the defect and circumferentially expand within the defect to attach the delivery device to the defect. The dilation device may comprise an expandable ring adapted to fit within the defect and yet still allow an implant to be delivered through the bore and into the defect. In this instance, the delivery device preferably comprises means to expand and shrink the dilation device. The dilation device may for example be expanded using gas or liquid. The delivery device may comprise a pump, which is preferably adapted to safely inflate the dilation device in a controlled manner. In an alternative embodiment, a separate pump may be used to inflate/expand the dilation device.

The attachment may comprise at least one guide wire, the/or each guide wire defining a distal and a proximal end and being suitable for insertion into a defect or about a defect. The guide wire may be attached to the delivery device or it may be attachable to the delivery device, or it may be separate from it. The guide wire may be threaded at its distal end to facilitate insertion into and removal from the defect by a user. The guide wire may be threaded at its proximal end for attachment to a rotation device and/or a handle. The rotation device may be a manually operated or power operated drill. The guide wire is preferably rigid and resistant to bending, and more preferably comprises metal, for example steel. The guide wire may be between 0.5-5 mm in diameter, and more preferably 1-2 mm in diameter.

In other embodiments, the attachment may comprise one or more guide wires, which may be attachable to the defect, or the site surrounding the defect. Each guide wire may be attachable to the defect or the site surrounding the defect for example by using surgical suturing.

The attachment may be removable from the barrel or may be integral with it. The attachment may be disposable or re-useable.

In further embodiments, the barrel of the delivery device may comprise means to provide friction-retarded movement of other surgical instruments adapted to fit within the bore of the delivery device. In one embodiment the barrel comprises serrated teeth within the bore, the teeth being adapted to frictionally engage with teeth positioned on other surgical instruments, and thereby limit any unwanted movement. In an alternative embodiment the barrel comprises a series of ridges or beads adapted to frictionally engage with beads or ridges positioned on other surgical instruments. Other means of frictionally limiting movement between objects in a sliding relationship are envisaged and will be known to a person skilled in the art.

Medical Implant Properties

The implant may comprise a liquid absorbed within it, which may be the same or different from the liquid in the barrel.

The implant may comprise a porous or non-porous material. Preferably, the implant comprises a porous biomaterial. The implant may be inelastic. The device can therefore be used with non-porous and/or inelastic implants. In the situation where the implant is inelastic, the delivery device must be appropriately sized to accommodate and deliver the implant without providing substantial stress on the implant, which cannot deform in response to the stress.

In particular embodiments the implant comprises a porous biomaterial, for example as described in PCT/GB04/004550, PCT/US2005/033873, PCT/GB2006/000797, and PCT/GB2007/003046, which may be a substantially elastic or viscoelastic biomaterial, for example as described in GB0721158.4.

Macroporosity typically refers to features associated with pores on the scale of greater than approximately 10 microns. Microporosity typically refers to features associated with pores on the scale of less than approximately 10 microns. It will be appreciated that there can be any combination of open and closed cells within the material. For example, the implant material will generally contain both macropores and micropores. The macroporosity is generally open-celled, although there may be a closed cell component.

The macropore size range (pore diameter) in the implant material may be from 1 to 1200 microns, preferably from 10 to 1000 microns, more preferably from 100 to 800 microns, still more preferably from 200 to 600 microns.

The mean aspect ratio range in the implant material may be from 1 to 50, more preferably from 1 to 10, and most preferably approximately 1.

The pore size distribution (the standard deviation of the mean pore diameter) in the implant material may be from 1 to 800 microns, more preferably from 10 to 400 microns, and still more preferably from 20 to 200 microns.

The porosity in the implant material may be from 50 to 99.99 vol %, and more preferably from 70 to 98 vol %.

The percentage of open-cell porosity (measured as a percentage of the total number of pores both open- and closed-cell) in the implant material may be from 1 to 100%, more preferably from 20 to 100%, and still more preferably from 90 to 100%. The implant material may be characterized by a progressively changing pore volume fraction, ranging from a pore fraction of 0 to 0.999.

Implant materials, such as bio-materials, that are non-uniformly porous are especially suited for tissue engineering, repair or regeneration, wherein the tissue is a connector tissue, or wherein the biomaterial is utilised to engineer, repair or regenerate two or more tissues in close proximity to one another. A difference in porosity may facilitate migration of different cell types to the appropriate regions of the biomaterial. A difference in porosity may facilitate development of appropriate cell-to-cell connections among the cell types comprised within the biomaterial, required for appropriate structuring of the developing/repairing/regenerating tissue. For example, dendrites or cell processes extension may be accommodated more appropriately via the varied porosity of the biomaterial. In particular embodiments, the permeability differences in the implant material may prevent and enhance penetration of, for example, liquids and other substances, wherein penetration is a function of molecular size, such that the lack of uniform porosity serves as a molecular sieve.

The implant material may comprise regions devoid of pores. The regions may be impenetrable to molecules with a radius of gyration or effective diameter of at least 1000 Daltons in size. The implant material may vary in its average pore diameter, or pore size distribution, concentration of components, cross-link density, or a combination thereof.

The biomaterial may be used to fabricate, for example, a porous monolithic scaffold, or a multi-layered scaffold in which at least one layer is porous. The biomaterial according to the present invention is advantageously used as a tissue regeneration scaffold for musculoskeletal and dental applications.

The biomaterial may be a composite biomaterial. Yet more preferably the implant comprises or may be a substantially elastic or viscoelastic porous biomaterial, the biomaterial comprising at least one selected from the group consisting of: a synthetic or natural polymer, a ceramic, a metal, an extracellular matrix protein, a calcium phosphate material or an analogue thereof.

The extracellular matrix protein may comprise collagen, one or more glycosaminoglycans, or a combination thereof. The biomaterial may further comprise separate or integrated calcium and phosphate sources, which is preferably a calcium phosphate material. The extracellular matrix protein may comprise hyaluronic acid and/or its salts, such as sodium hyaluronate; mucinous glycoproteins (e.g. lubricin), vitronectin, tribonectins, surface-active phospholipids, rooster comb hyaluronate. Hyaluronic acid may be derived from human umbilical chord. The extracellular matrix protein may also comprise heparin, for example, derived from porcine intestinal mucosa.

The extracellular matrix proteins may be purified from tissue, by means well known in the art. For example, if collagen is used, the naturally occurring extracellular matrix can be treated to remove substantially all materials other than collagen, for example glycoproteins, glycosaminoglycans, proteoglycans, lipids, non-collagenous proteins and nucleic acid (DNA or RNA), by known methods.

The collagen may be Type I collagen, Type II collagen, Type IV collagen, gelatin, agarose, cell-contracted collagen containing proteoglycans, glycosaminoglycans or glycoproteins, fibronectin, laminin, elastin, fibrin, synthetic polymeric fibers made of poly-acids such as polylactic, polyglycolic or polyamino acids, polycaprolactones, polyamino acids, polypeptide gel, copolymers thereof and/or combinations thereof.

In this context, the term “copolymer” refers to a material comprising two or more distinct polymer species, wherein at least some chemical bonding between the two or more distinct polymer species is present. Preferably at least one of the distinct polymer species comprises a type of collagen. Similarly, the term “combination” refers to a material comprising two or more distinct polymer species, wherein no chemical bonding between the two or more distinct polymer species is present and preferably where at least one of the distinct polymer species comprises a type of collagen. The collagen may be soluble or insoluble and may be derived from any suitable tissue in any animal and may be extracted using any number of conventional techniques.

The polymer may be inorganic, yet be biocompatible, and may be hydroxyapatite, all calcium phosphates, alpha-tricalcium phosphate, beta tricalcium phosphate, calcium carbonate, barium carbonate, calcium sulphate, barium sulphate, polymorphs of calcium phosphate, ceramic particles, or combinations thereof.

Glycosaminoglycans are a family of macromolecules containing long unbranched polysaccharides containing a repeating disaccharide unit. Preferably, the one or more glycosaminoglycans are selected from dermatan sulphate, heparan sulphate, heparin, chondroitin sulphate and/or keratan sulphate. Chondroitin sulphate may be chondroitin-4-sulphate or chondroitin-6-sulphate, both of which are available from Sigma-Aldrich Inc. The chondroitin-6-sulphate may be derived from shark cartilage.

Thus the biomaterial may comprise collagen, one or more glycosaminoglycans and a calcium phosphate material.

The calcium source is preferably selected from one or more of calcium nitrate, calcium acetate, calcium chloride, calcium carbonate, calcium alkoxide, calcium hydroxide, calcium silicate, calcium sulphate, calcium gluconate and the calcium salt of heparin. A calcium salt of heparin may be derived from the porcine intestinal mucosa. Suitable calcium salts are commercially available from Sigma-Aldrich Inc.

The phosphorus source is preferably selected from one or more of ammonium-dihydrogen phosphate, diammonium hydrogen phosphate, phosphoric acid, disodium hydrogen orthophosphate 2-hydrate Na₂HPO_{4.}2H₂O, sometimes termed GPR Sorensen's salt) and trimethyl phosphate, alkali metal salts (e.g. Na or K) of phosphate, alkaline earth salts (e.g. Mg or Ca) of phosphate.

At least part of the biomaterial may be formed from a porous co-precipitate comprising a calcium phosphate material and one or more of collagen (including recombinant human (rh) collagen), a glycosaminoglycan, albumin, hyaluronan, chitosan, or a synthetic polypeptide comprising a portion of the polypeptide sequence of collagen.

At least part of the biomaterial may be formed from a porous co-precipitate comprising a calcium phosphate material and collagen.

At least part of the biomaterial may be formed from a porous triple co-precipitate comprising a calcium phosphate material and two or more of collagen (including recombinant forms such as recombinant human (rh) collagen), a glycosaminoglycan, albumin, hyaluronan, chitosan, and a synthetic polypeptide comprising a portion of the polypeptide sequence of collagen.

Preferably, at least part of the biomaterial is formed from a porous triple co-precipitate comprising collagen, a glycosaminoglycan and a calcium phosphate material.

The applicant's earlier application, PCT/GB04/004550, filed 28 Oct. 2004, describes a triple co-precipitate of collagen, brushite and a glycosaminoglycan and a process for its preparation. The content of PCT/GB04/004550 is incorporated herein by reference. The process described in PCT/GB04/004550 involves: providing an acidic aqueous solution comprising collagen, a calcium source and a phosphorous source and a glycosaminoglycan; and precipitating the collagen, the brushite and the glycosaminoglycan together from the aqueous solution to form a triple co-precipitate.

The term co-precipitate encompasses precipitation of compounds where the compounds have been precipitated at substantially the same time from the same solution/dispersion. It is to be distinguished from a material formed from the mechanical mixing of the components, particularly where these components have been precipitated separately, for instance in different solutions. The microstructure of a co-precipitate is substantially different from a material formed from the mechanical mixing of its components. Preferably the co-precipitate is a triple co-precipitate of three compounds (tri-precipitate). It will be apparent to the skilled person however, that a co-precipitate may comprise further components that may also be co-precipitated for example tetra, penta, hexa, hepta, octa, nona or deca-precipitates or components that are combined with the co-precipitate, for example, using mechanical or chemical means.

Thus, at least part of the biomaterial may be formed from a porous triple co-precipitate comprising collagen, a glycosaminoglycan and a calcium phosphate material. The calcium phosphate material may be brushite, hydroxyapatite, octacalcium phosphate, or combinations thereof. The glycosaminoglycan may be a chondroitin sulphate. The collagen and the one or more glycosaminoglycans may be crosslinked.

The biomaterial may be a gradient biomaterial. The biomaterial may be non-uniformly porous. Non-uniform porosity may allow for permeability at some regions and not others, within the biomaterial, or the extent of permeability may differ within the biomaterial.

The pores within such a gradient biomaterial may be of a non-uniform average diameter. The average diameter of the pores may range from 0.001-1500 microns. The average diameter of the pores may vary as a function of its spatial organization in the biomaterial. The average diameter of the pores may vary along an arbitrary axis of the biomaterial.

The biomaterial may comprise one or more pharmacological agents or biomolecules, or combinations thereof. The biomolecules may be selected from the group consisting of: cytokines, growth factors, hormones or combinations thereof. Preferred biomolecules include growth hormone (GH), parathyroid hormone (PTH), Calcitriol, Osteoprotegerin, calcitonin, thyroid stimulating hormone (TSH) and Leptin. The pharmacological agent may be any agent, although it is envisaged that the most useful agents will be those that e.g. promote healing, prevent infection, reduce inflammation, minimise or prevent pain, stimulate the influx of healing cells, or act as an immunosuppressant.

The biomaterial may vary in its concentration or distribution of biomolecules and/or pharmacological agents. The biomaterial may vary in terms of its polymer concentration, or concentration of and component of the biomaterial, including biomolecules and/or cells incorporated within the biomaterial. The biomaterial may vary along any given direction in the concentration of its components, cross-link density, or a combination thereof.

The concentration of the polymer in the biomaterial may vary as a function of its spatial organization in the biomaterial. The concentration may vary along a given direction in the biomaterial. The crosslink density of the biomaterial may vary along a desired direction in the biomaterial.

In preferred embodiments, the biomaterial is a substantially elastic or viscoelastic composite biomaterial comprising: a first layer formed of a biomaterial as described above, and a second layer joined to the first layer and formed of a material comprising collagen, or a co-precipitate of collagen and a glycosaminoglycan, or a co-precipitate of collagen and a calcium phosphate material, or a triple co-precipitate of collagen, a glycosaminoglycan and a calcium phosphate material. Preferably the biomaterial is under stress and is in a fully or partially-deformed state.

The first layer may be formed of a porous material comprising collagen and a calcium phosphate material and optionally a glycosaminoglycan.

The first and second layers may be integrally formed, preferably by liquid phase co-synthesis. The first and second layers may be joined to one another through an inter-diffusion layer.

Advantageously, this may be achieved by a process involving liquid phase co-synthesis. This encompasses any process in which adjacent layers, either dense or porous, of a material comprising multiple layers are formed by placing slurries comprising the precursors to each layer in integral contact with each other before removal of the liquid carrier or carriers from the slurries, and in which removal of the liquid carrier or carriers from all layers is preferably performed at substantially the same time. Placing the precursor slurries in integral contact before removal of the liquid carrier (i.e. while still in the liquid phase) allows interdiffusion to occur between adjacent slurries. This results in a zone of interdiffusion at the interface between adjacent layers of the resulting material, within which the material composition is intermediate to the material compositions of the adjacent layers. The existence of a zone of interdiffusion can impart mechanical strength and stability to the interface between adjacent layers. Accordingly, the first and second layers are preferably joined to one another through an inter-diffusion layer.

The first and second layers may be joined to one another through an inter-layer. The term inter-layer refers to any layer deposited independently between two other layers for the purpose of improving inter-layer bond strength or blocking the passage of cells, molecules or fluids between adjacent layers of the resulting biomaterial, and may, for example, contain collagen, glycosaminoglycans, fibrin, anti-angiogenic drugs (e.g. suramin), growth factors, genes, biomolecules, pharmacological agents, or any other constituents. An inter-layer is distinguished from an inter-diffusion layer by the fact that an inter-layer is deposited separately as a slurry whose composition is distinct from the composition of its adjacent layers, while an inter-diffusion layer is formed exclusively as a result of inter-diffusion between adjacent layers.

The second layer may be porous or non-porous.

The biomaterial may comprise one or more further layers joined to the first and/or second layers, each of the further layers being formed of a material comprising collagen, or a co-precipitate of collagen and a glycosaminoglycan, or a co-precipitate of collagen and a calcium phosphate material, or a triple co-precipitate of collagen, a glycosaminoglycan, and at least one calcium phosphate material.

The first and second layers and the one or more further layers may be integrally formed. Adjacent layers may be joined to one another through an inter-diffusion layer.

At least one of the one or more further layers may be porous or non-porous.

The macropore size range (pore diameter) in the porous material described above is also applicable to second and/or further layers. The same is true for the mean aspect ratio range, the pore size distribution, the porosity and the percentage of open-cell porosity.

Differences in pore sizes between adjacent layers may vary from almost negligible to as great as +/−1000 microns.

The biomaterial preferably comprises collagen and a glycosaminoglycan, which is preferably a chondroitin sulphate. The collagen and glycosaminoglycan may be crosslinked.

The biomaterial may comprise collagen in an amount of from 1 to 99 wt %, preferably from 5 to 90 wt %, more preferably from 15 to 60 wt %.

The glycosaminoglycan may be present in the material in an amount of from 0.01 to 20 wt %, preferably from 1 to 5.5 wt %.

The biomaterial may comprise collagen, and if the calcium phosphate comprises brushite, the ratio of collagen to brushite is from 10:1 to 1:100 by weight, preferably from 5:1 to 1:20 by weight.

The biomaterial may comprise collagen, and if the calcium phosphate material comprises octacalcium phosphate, the ratio of collagen to octacalcium:phosphate is from 10:1 to 1:100 by weight, preferably from 5:1 to 1:20 by weight.

The biomaterial may comprise collagen and a glycosaminoglycan, wherein the ratio of collagen to the glycosaminoglycan is from 8:1 to 30:1 by weight.

Thus, the elastic and viscoelastic properties of the implant material are influenced by several variables. The present inventors have determined that the ability of a biomaterial to strain under stress is influenced by inter alia one or more of: the hydration status of the biomaterial, the degree of cross-linking, and various aspects relating to the pores contained within the biomaterial, for example the pore size, pore structure, pore shape, pore uniformity.

The elastic and viscoelastic properties of implants comprising biomaterials or other materials may therefore be tailored by varying one or more of these parameters, which may allow an e.g. particularly elastic material to be used to treat a particular type or size or position of defect, whereas a less elastic material may be required to treat other types, sizes, or positions of defects. For implants comprising two or more layers, or a gradient within one or more layers, it is envisaged that the elastic/viscoelastic properties could vary within and between the one or more layers.

The biomaterial may be used to fabricate, for example, a porous monolithic scaffold, or a multi-layered scaffold in which at least one layer is porous. The composite biomaterial according to the present invention is advantageously used as a tissue regeneration scaffold for musculoskeletal and dental applications.

Multilayer (i.e. two or more layers) scaffolds according to the present invention may find application in, for example, bone/cartilage interfaces (e.g. articular joints), bone/tendon interfaces (e.g. tendon insertion points), bone/ligament interfaces (e.g. ligament insertion points), and tooth/ligament interfaces (e.g. tooth/periodontal ligament juncture).

The biomaterial may be used to fabricate implants that persist in the body for quite some time. For example, a semi-permanent implant may be necessary for tendon and ligament applications.

As can be seen from the previous description, the present invention provides processes and methods by which an implant can be prepared for implantation, or positioned within, and discharged from, a delivery device. The invention further provides a device suitable for employing these processes and methods. The advantage of using implant contacting means is that the implant is not damaged or stressed in an undesirable way. The use of liquid in combination with porous biomaterials is particularly advantageous since this also overcomes the problem of dehydration of the tissues surrounding the defect site.

The present invention will now be described further by way of example, and with reference to the drawings. These examples are provided to further assist in the understanding of the present invention, and are not to be considered limiting to the scope of the invention. Any feature described in the examples is applicable to any aspect of the foregoing description.

### BRIEF DESCRIPTION OF FIGURES

FIG. 1 shows an implant delivery device and its component parts in isometric projection;

FIG. 2 is a series of longitudinal sections through the device of FIG. 1 showing the steps involved in the multi-stage actuation of the device;

FIG. 3 shows an implant delivery device where FIG. 3a shows the device in side view, and FIG. 3b is a longitudinal section of the barrel of the device along the axis B-B; and

FIG. 4 is a series of longitudinal sections of the device showing the stages in the movement and discharge of an implant through the barrel of the device and out of the distal end, where FIG. 4a shows the device with an implant loaded in the proximal end of the barrel, the barrel containing fluid and with a plunger positioned within it, FIG. 4b shows the device with the plunger partially depressed and the implant moving through the neck/taper of the distal tip, the neck causing the distal most portion of the biomaterial within the implant to deform toward its partially/fully deformed state; FIG. 4c shows the implant now fully positioned within the distal tip, the entire implant now being deformed toward its partially/fully deformed state; and FIG. 4d shows the deformed implant prior to being discharged through the distal end of the device.

FIG. 5a is a photograph showing a porous biomaterial comprising a triple co-precipitate of collagen, glycosaminoglycan and calcium phosphate saturated with liquid (a red dye);

FIG. 5b is a photograph showing the porous biomaterial from FIG. 5a in longitudinal section.

The examples, materials, procedures, drawings, figures, references and content of PCT/GB04/004550, PCT/US2005/033873, PCT/GB2006/000797, PCT/GB2007/003046 and GB0721158.4 are incorporated herein by reference.

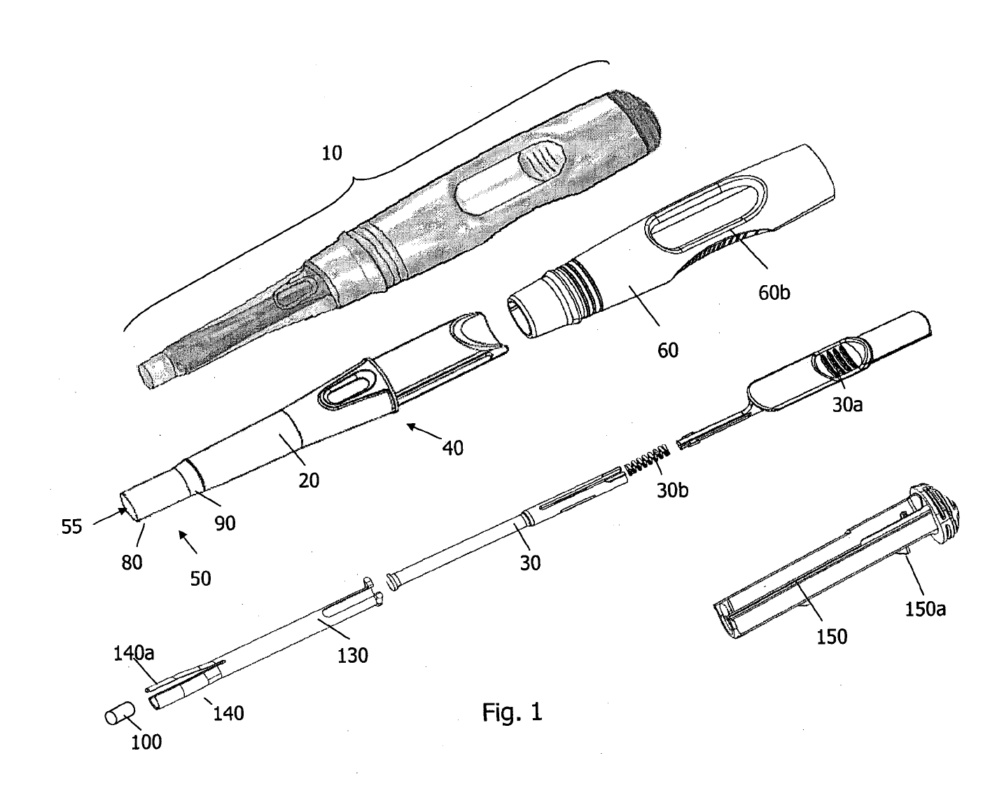

Referring to FIG. 1, the implant delivery device (10) comprises a barrel (20), a plunger (30) and an inner sleeve (130) which comprises material stress means (140), in this instance, in the form of three longitudinally extending arms (140a). The barrel defines a proximal end (40), a distal end (50) and a bore (55). The plunger (30) is adapted to fit into a central bore of the inner sleeve (130) in a sliding relationship and the inner sleeve (130) in turn is adapted to fit into the bore of the barrel (20) also in a sliding relationship. The barrel comprises a handle (60) at its proximal end (40) being adapted to engage with the hand and/or fingers of a user. The plunger (30) and inner sleeve (130) further comprise actuation means (30a and 150 respectively) adapted to engage with both the handle (60) of the device and with the thumb and/or fingers of a user to move either the inner sleeve (130) and/or plunger (30) within the barrel.

The barrel comprises a neck region (90) where the diameter of the barrel (20) gradually tapers to a distal tip (80) having a diameter narrower than that of the barrel (20). In the embodiment shown the barrel (20) of the implant delivery device (10) is straight however, in other embodiments the barrel can be, or may comprise, regions (flexible or otherwise) that are curved or bent or which may be adjusted for a desired degree of bend or curvature.

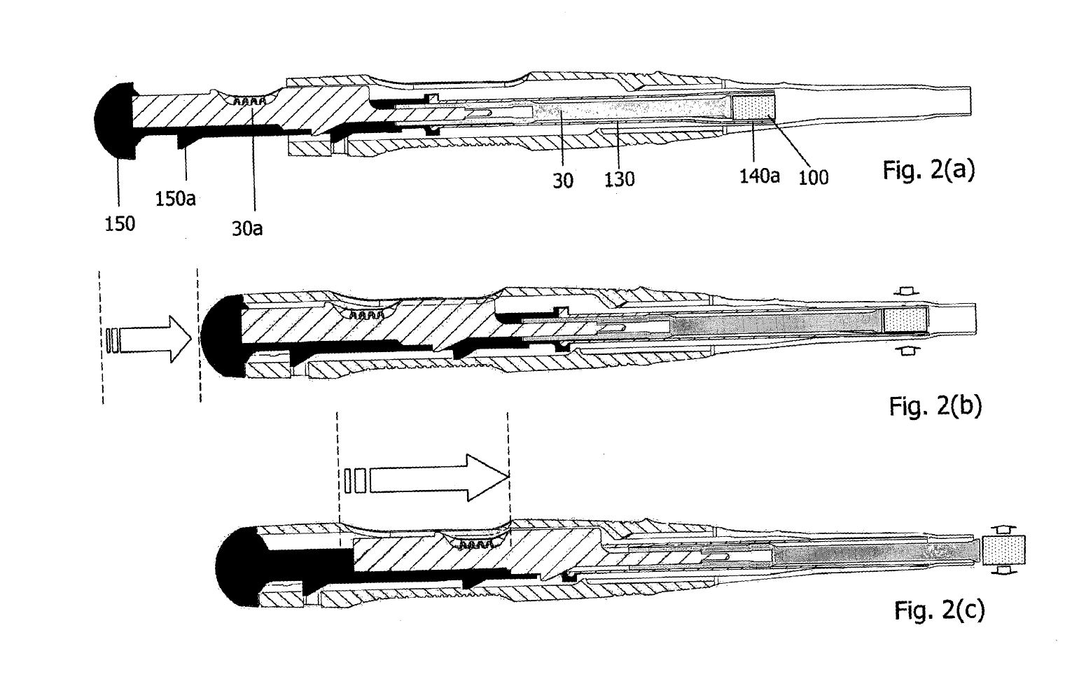

FIG. 2a shows an implant (100) positioned within implant contacting means, arms (140a) of the inner sleeve (130) of the device ready to be actuated by a user. The implant (100) comprises a biomaterial (120) that is substantially elastic or viscoelastic and which can be deformed through the application of stress from a resting position to a partially or fully deformed state. The implant (100) can comprise one or more layers of biomaterial (i.e. a composite or layered implant), each of which can be tailored to suit the physical and chemical characteristics of the tissue which it is designed to replace.

In use, a user first depresses or actuates the inner sleeve actuator (150), FIG. 2b. As the inner sleeve actuator (150) travels longitudinally within the handle in the direction of the distal tip (80) of the barrel, the inner sleeve (130) including the plunger (30) and compression actuator (30a) also move in the direction of the distal tip (80). The inner sleeve actuator (150) completes its range of movement when it abuts the end of the handle (60) and is locked in position by a shaped projection (150a) that engages a correspondingly shaped groove or hole (60a) within the handle.

As the user depresses the inner sleeve actuator (150), the inner sleeve (130) moves within the barrel (20) towards the distal end (50). As the inner sleeve (130) moves, it carries the implant (100) within it, held between the arms (140a) of the inner sleeve (130). As the arms (140a) of the inner sleeve (130) travel along the tapered neck region (90) of the barrel (20), the decreased/decreasing diameter of the barrel (20) applies stress on the arms (140a) of the inner sleeve (130) urging the arms radially inwardly, i.e. toward the longitudinal axis of the barrel. In turn the arms (140a) apply stress on the implant (100) causing the biomaterial (120) in the implant (100) to deform or compress towards its partially or fully deformed state. Movement of the inner sleeve actuator (150) and respective movement of the inner sleeve (130) positions the implant at the distal tip (80) of the device (10) in a partially or fully deformed state in readiness for discharge of the implant (100) from the device into the cavity or hole.

In the next stage, shown in FIG. 2c the user simply depresses the compression actuator (30a) which in turn exerts a force on the plunger (30) moving the implant (100) distally along and out of the inner sleeve (130) and distal tip (80) of the barrel. The compression actuator (30a) completes its range of movement when it abuts the base of an opening (60b) in the handle (60) and is also locked in position to prevent further movement. By delivering an implant (100) in its partially or fully deformed state, a user can position the implant (100) in a defect site and allow the elastic/viscoelastic properties of the implant (100) to expand toward its rest state and in so doing, completely fill the defect site. The implant being in a partially or fully deformed state will expand toward the rest state immediately or over a period of time depending on the elastic/viscoelastic properties of the chosen implant material.

In the event that the distal tip (80) of the barrel is blocked or covered such that the implant (100) is unable to be discharged from the device (10), the biasing means, in this case in the form of a spring (30b), compresses to enable the compression actuator (30a) to complete its full range of motion and become locked in its final position. The compressed spring (30b) maintains a force on the plunger (30) in the direction of the distal tip (80) such that as soon as the opening at the distal tip (80) of the barrel (20) is free from obstruction, the compressed spring will expand pushing the plunger (30) in a longitudinal direction toward the distal tip (80) of the barrel (20). This movement in turn pushes the implant (100) out of the arms of the inner sleeve (130) and distal tip (80) of the device in a relatively short space of time. Thus, a user may position the distal tip (80) of the device (10) at the base of a cavity or hole and then actuate the inner sleeve actuator (150) and compression actuator (30a) in turn. As the device (10) is removed from the cavity or hole, the implant (100) is simultaneously discharged/ejected from the distal tip (80) of the device (10) into the cavity or hole. There the implant (100) expands to its rest state thereby filling the cavity or hole. In this instance the implant maintains its position within the cavity or hole by friction fit.

In an alternative embodiment, the implant is not held or grasped by the arms (140a), rather, the arms are urged inwardly upon actuation by a user such that they only contact the proximal end of the implant, which is then pushed along the barrel during the discharging procedure, essentially as described.

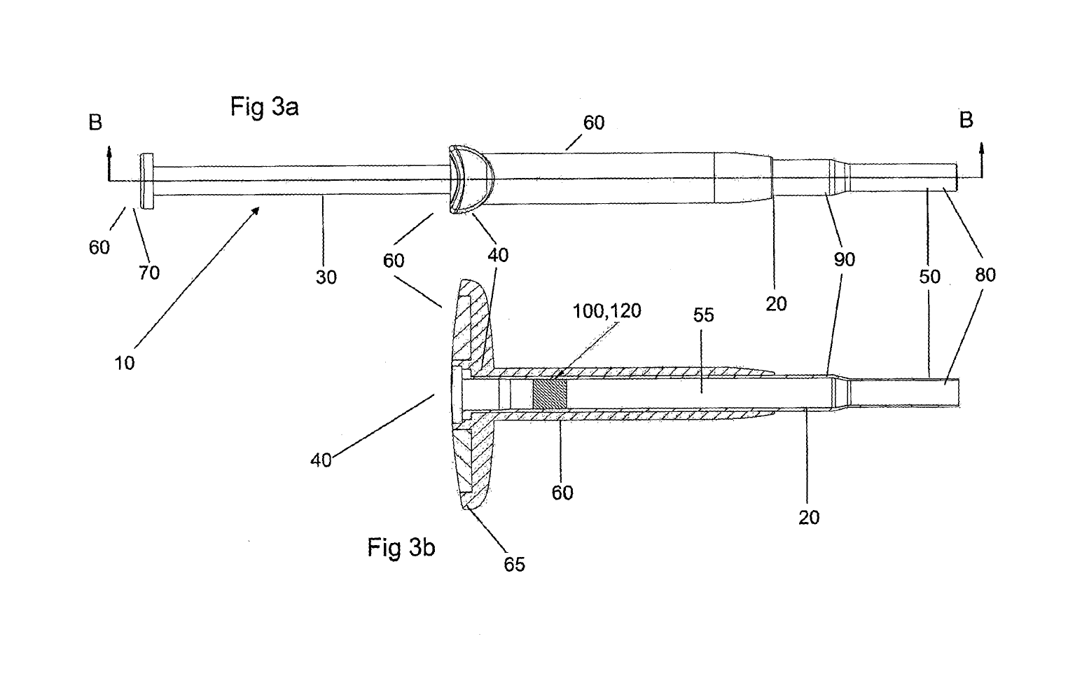

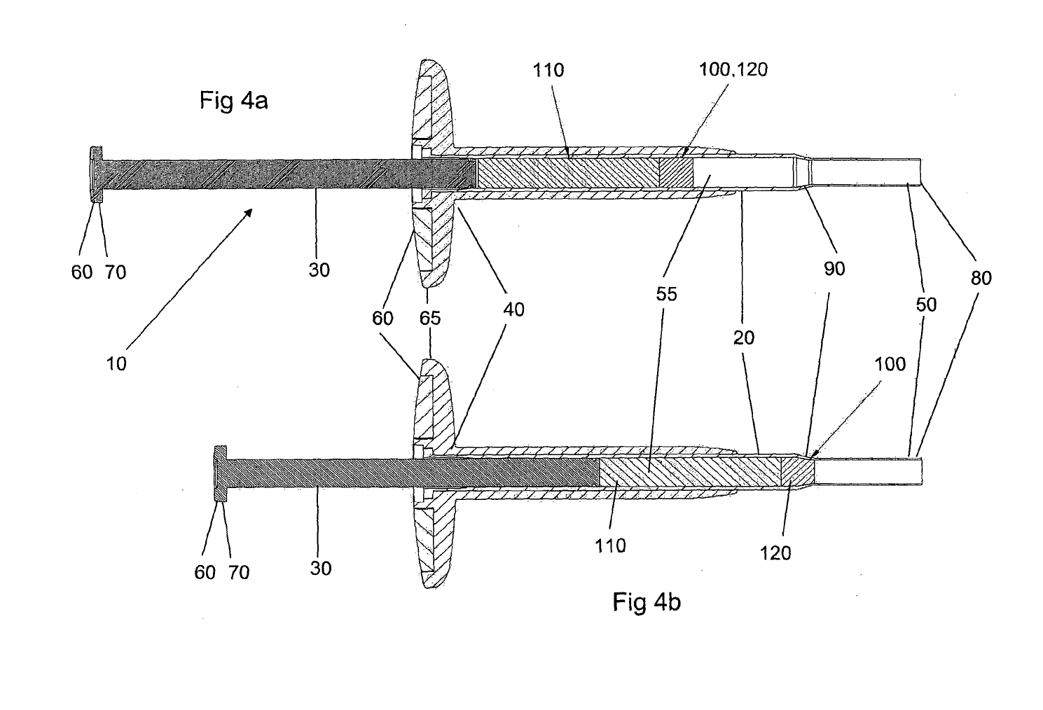

FIGS. 3 and 4 depict an alternative embodiment of the device (10). The implant delivery device (10) generally comprises a barrel (20) and plunger (30), the barrel defining a proximal end (40) and distal end (50), and bore (55). The plunger (30) is adapted to fit into the bore of the barrel (20) in a sliding relationship. The barrel comprises a handle (60), taking the form of laterally extending projections (65) adapted to engage with the fingers of a user. The plunger similarly comprises a handle (60) which is in the form of a flat disc (70), adapted to engage with the thumb or fingers of a user depending on whether the plunger (30) is being respectively pressed or pulled by a user. The barrel comprises a distal tip (80) having a diameter narrower than that of the barrel (20), with barrel comprising a neck region (90) where the diameter of the barrel (20) gradually tapers to that of the distal tip (80). In the embodiment shown, the barrel (20) of the implant delivery device (10) is straight; however in other embodiments, the barrel can be, or can comprise regions (flexible or otherwise) which are curved or bent, or which can be adjusted for a desired degree of bend or curvature.

An implant (100) is shown (FIG. 3b) positioned within the barrel (20). The implant (100) comprises a biomaterial (120), which is substantially elastic or viscoelastic and which can be deformed, through the application of stress, from a resting position to a partially or fully deformed state. The implant (100) can comprise one of more layers of biomaterial (120) (a composite or layered implant), each of which can be tailored to suit the physical and chemical characteristics of the tissue which it is designed to replace.

In FIG. 4a the implant delivery device (10) is shown with an implant (100) loaded within the barrel (20), and a column of liquid (implant contacting means, 110) located proximally behind the implant (100). In preferred embodiments, the liquid (110) lies in front of the implant (100), i.e. distally as well as proximally, although this is not shown in the Figures for reasons of clarity. Plunger (30) is positioned within the barrel (20), ready to be actuated by a user. Where the liquid (110) is positioned within the entire barrel (20) the implant (100) can be fully or partially immersed within it.

A user depressing (or actuating) plunger (30) exerts a compression force on liquid (110), which is transferred onto the implant (100) because of the substantially incompressible nature of the liquid (110). Thus, as the user depresses the plunger (30) into the barrel (20), the implant (100) moves distally along the barrel (20) toward the distal end (50). In certain embodiments (not shown) the implant delivery device comprises liquid addition means and/or liquid retention means. The liquid addition means can be a tube, attached or attachable to the proximal end or the barrel of the device and by which liquid can be added into the barrel of the device. In alternative embodiments the liquid addition means can be a tube or cannula, and which can pass through plunger (30). Liquids can be added into the barrel, thereby allowing a user to contact an implant with liquid in the barrel, or “top up” liquid in the barrel, to maintain a desired volume. Liquids with differing compositions can be added e.g. sequentially, and mixed in situ thereby allowing a user to load the device with an implant (which can be dry or pre-soaked), and add one or more liquids thereafter. In this way, the liquid within which a pre-soaked implant can be stored can be changed following loading of the implant into the barrel, and addition and mixing of one or more further liquids.

The liquid retention means (not shown) can take the form of a valve, barrier, plug, stopper, sheath, or tap, and which can be used to prevent liquid discharging from the device. In its simplest form the liquid retention means can comprise the finger of a user placed over the distal tip. In order to allow the implant to move distally along the barrel and discharge out of the device, a user will have to disengage the liquid retention means in order to let liquid discharge from the device. A user can simply remove their finger or any barrier, plug, stopper, or sheath or disengage any valve or tap.

The biomaterial (120) within the implant (100) can vary in terms of its physical and chemical properties, and the pore size of the biomaterial (120) will govern to some extent the ability of liquid (110) to pass through the implant (100) as it moves within the barrel. With some implants the liquid (110) may not pass through the implant (100), and in other versions, some of the liquid (110) may pass through the implant (100) as it travels along the barrel (20). Even if fluid does pass through the implant (100), the resistance of the implant (100) to being totally “flushed through” means that at least some of the compression force on the liquid (110) will be converted to movement of the implant (100) within the barrel (20).

In the embodiment shown in FIG. 4, the implant (100) is travelling distally through the barrel (20); however, the implant (100) can be moved in a proximal direction by a user partially or fully pulling the plunger (30) out of the barrel, thereby creating a flow of liquid in a proximal direction, the flow causing movement of the implant (100) along the barrel (20) in the desired direction. In this way an implant (100) can be moved proximally and distally within the barrel (20), thereby enabling a user to position the implant (100) at any desired point within the barrel (20). To facilitate positioning and discharge of an implant (100) from the device (10), the barrel, or at least part of the barrel is completely transparent or translucent. In other embodiments (not shown), the barrel (20) can comprise one or more windows, and/or only the distal end (50) is transparent or translucent. In some embodiments the barrel (20) preferably comprises markings (not shown) which enable a user to quickly determine the size of the bore (55). The barrel can be coloured or tinted. Any of these features can be useful where a number of barrels (20) or implant delivery devices (10) are comprised within a kit where a user selects an appropriately sized barrel for an implant or defect to be treated. In some embodiments the barrel (20) preferably comprises markings which enable a user to quickly determine the depth of insertion of the distal tip (80) of the device (10) into a defect site.

Referring to FIG. 4b, the implant (100) is deformed as it travels along the tapered neck region (90) of the barrel (20), whereby the decreased diameter of the barrel applies stress on the implant (100) causes the biomaterial (120) in the implant (100) to deform toward its partially or fully deformed state. By compressing the implant (100), the pore size within the biomaterial (120) is reduced, which can reduce the amount of liquid (110) passing through the implant (100), thereby increasing the degree of movement of the implant (100) within the barrel (20), relative to the implant (100) in its non-deformed (rest) state. By delivering an implant (100) in its partially or fully deformed state, a user can position the implant (100) in a defect site, and allow the elastic/viscoelastic properties of the implant (100) to expand it toward its rest state, and in doing so, completely fill the defect site, thereby facilitating healing of the defect and maintenance of the implant (100) within the site.

In alternative embodiments of the device, the diameter of the barrel and/or distal end is adjustable, thereby enabling a user to vary the amount of stress applied on the implant, and/or utilise different sized implants within the same barrel. In these embodiments, preferably the stress can be applied either uniformly, for example radially at a cross-sectional point or locally, causing one region of the implant to deform with respect to another region.

FIG. 4c shows the implant delivery device (10) with the plunger (30) more fully depressed within the barrel (20). The implant (100) has now fully traversed the tapered neck region (90), and resides within the distal tip (80) in a deformed state (whether partially or fully so).

In the final stage (FIG. 4d) the implant (100) is positioned ready for discharge from the implant delivery device (10) into the defect site. A user need simply further depress the plunger (30) and the implant (100) will exit the distal tip (80) and out of the device (10). If the implant is in a partially or fully deformed state, the implant will expand toward the rest state either immediately over a time period, depending on the elastic/viscoelastic properties of the chosen biomaterial.

A. Synthesis of a Porous Biomaterial

A porous biomaterial comprising a triple co-precipitate of collagen, glycosaminoglycan and calcium phosphate was synthesised according to Example 3 of PCT/GB04/004550. The elastic/viscoelastic properties of this material are described in GB0721158.4.

B. Absorption of Liquid by the Biomaterial

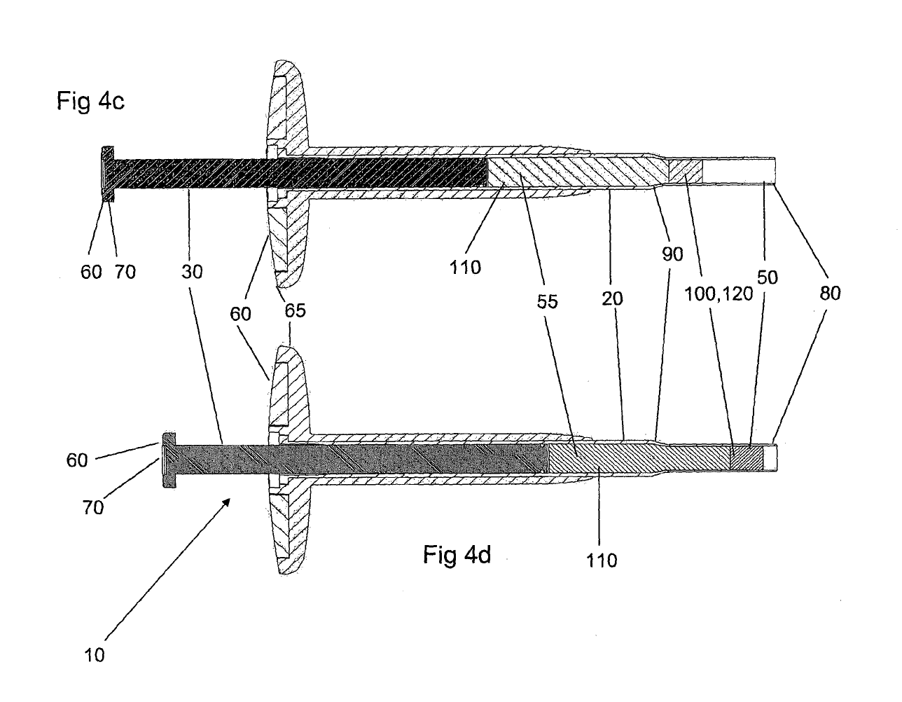

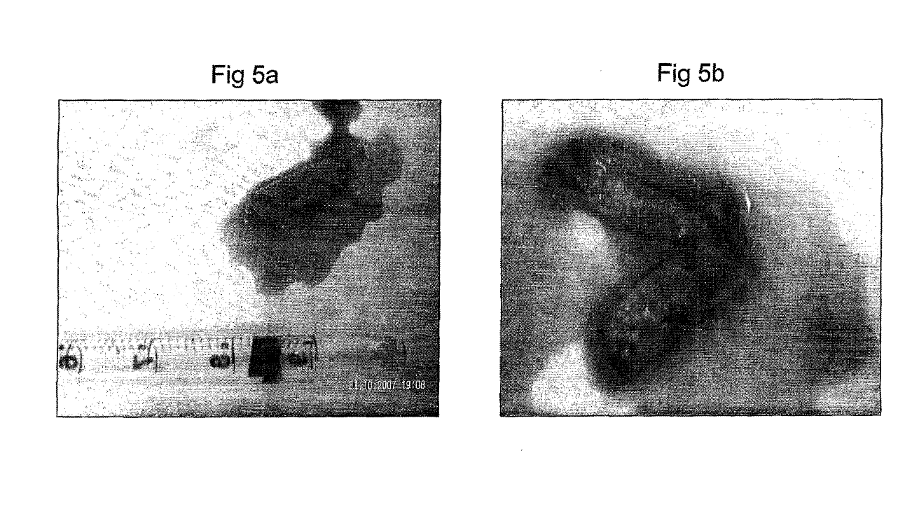

The porous biomaterial was fashioned into a cylindrical plug adapted to fit within the bore of a glass pipette. The ability of the biomaterial to absorb liquid was investigated by placing the biomaterial in a volume of red dye. The biomaterial absorbed the dye at a uniform rate, and appeared to be fully saturated within 5 minutes (FIG. 5). To confirm this, the biomaterial containing the absorbed dye was then sectioned longitudinally (FIG. 6). The sectioned biomaterial clearly showed that the red dye had been uniformly absorbed throughout the material.

C. Manipulation of the Biomaterial in a Delivery Device

The porous biomaterial from Example A was fashioned into a cylindrical plug adapted to fit within the bore of a glass pipette, and was soaked in red dye, as in Example B. The pipette was filled with saline solution such that the biomaterial was fully immersed in the liquid. A compression force was applied to the liquid using positive pressure exerted with a pipette bulb. Liquid was discharged from the distal tip of the pipette, and the biomaterial moved with the flow of liquid through the bore of the pipette. The red dye remained absorbed in the biomaterial and was not flushed away by the saline solution.

D. Positioning of the Biomaterial in a Delivery Device

The aim of this experiment was to position the biomaterial at a desired distal position within the pipette, near the distal end. The biomaterial was prepared and loaded into a pipette as described in Example C. Upon application of a compression force using a pipette bulb, liquid was discharged from the distal tip of the pipette, and the biomaterial moved distally with the flow of liquid through the bore of the pipette. As the biomaterial approached the distal end of the pipette, the compression force was removed, and the volume of liquid discharging from the distal end reduced. At the same time, the biomaterial stopped moving within the saline solution and came to rest at the desired distal position. As with Example B, the red dye remained absorbed in the biomaterial and was not flushed away by the saline solution.

E. Discharge of the Biomaterial from a Delivery Device

The aim of this experiment was to discharge the biomaterial through the distal end of the pipette. The biomaterial was prepared, loaded and moved distally as described in Example D. The re-application of the compression force resulted in liquid being discharged from the distal tip of the pipette and the biomaterial moving distally toward the distal tip. As the biomaterial passed through the narrow neck of the pipette (shown in FIG. 5), the stress forces acting on the biomaterial caused it to deform, thereby allowing it to pass through the narrow aperture. As the biomaterial emerged from the device, it returned toward the non-deformed state. As with the previous Examples, the red dye remained absorbed in the biomaterial and was not flushed away by the saline solution.

In each of the above examples, liquid could be retained within the pipette by covering the distal end, e.g. with the finger of a user.

1. A delivery device suitable for implanting a medical implant into a cavity comprising:
(i) a barrel defining a bore and having a proximal end and a distal end, and
(ii) a plunger within the bore and being in slidable relationship with the barrel, characterised in that the delivery device further comprises:
(iii) implant contacting means positioned within the bore to hold an implant,
wherein, in use the implant contacting means is adapted to limit longitudinal deformation of an implant as it travels through the bore by eliminating, or reducing the duration of, direct contact between the plunger and the implant.

2-53. (canceled)

54. A delivery device suitable for implanting a medical implant into a cavity comprising:
(i) a barrel defining a bore and having a proximal end and a distal end, and
(ii) a plunger within the bore and being in slidable relationship with the barrel, characterised in that the delivery device further comprises:
(iii) implant contacting means positioned within the bore to hold an implant,
wherein, in use the implant contacting means is adapted to limit longitudinal deformation of an implant as it travels through the bore by eliminating, or reducing the duration of, direct contact between the plunger and the implant.

55. A delivery device as claimed in claim 54, further characterised in that the implant contacting means are adapted to cause radial deformation of an implant as the implant contacting means moves within the barrel.

56. A delivery device as claimed in claim 54, wherein the implant contacting means
comprises at least two arms, the arms being adapted to be urged inwardly toward the longitudinal axis of the bore of the barrel.

57. A delivery device as claimed in claim 54 wherein the implant contacting means are adapted to cause radial deformation of an implant upon actuation of the device by a user.

58. A delivery device as claimed in claim 54 wherein the device further comprises:
(iv) an inner sleeve having a proximal end and a distal end, wherein the distal end is operably linked to the implant contacting means, the inner sleeve being in slidable relationship with both the barrel and the plunger,
wherein in use the inner sleeve applies a force that is transferred to an implant to move the implant along the barrel in a distal direction.

59. A delivery device as claimed in claim 58 wherein, in use an implant is positioned distally from the plunger and wherein the inner sleeve is adapted to move the implant to a first position towards the distal end of the barrel and the plunger is adapted to move to a second position at the distal end of the barrel whereby movement of the plunger toward the second position pushes the implant out of the device.

60. A delivery device as claimed in claim 59 wherein the plunger is adapted to move together with the inner sleeve to the first position in the barrel.

61. A delivery device as claimed in claim 54 further comprising actuation means for initiating and/or assisting the discharge of an implant from the distal end of the barrel, the actuation means being in operative communication with the plunger and/or inner sleeve.

62. A delivery device as claimed in claim 61, wherein the actuation means is adapted to move the plunger and the inner sleeve to a first position and subsequently to move the plunger to a second position whereby in use movement to the first position causes deformation of an implant and movement toward the second position pushes the deformed implant out of the device.

63. A delivery device as claimed in claim 61 wherein the actuation means comprises first and second actuation means wherein, the first actuation means is adapted to move the second actuation means, plunger and where present inner sleeve to a first position and the second actuation means is adapted to move the plunger to a second position whereby in use, movement to the first position causes deformation of an implant and the movement toward the second position pushes the deformed implant out of the device.

64. A delivery device as claimed in claim 61 further comprising, first and second actuation means for initiating and/or assisting the discharge of an implant from the distal end of the barrel wherein, the first actuation means is adapted to move the second actuation means and inner sleeve to a first position and the second actuation means is adapted to move the plunger to a second position whereby in use, movement to the first position causes deformation of an implant and the movement toward the second position pushes the deformed implant out of the device.

65. A delivery device as claimed in claim 54 further comprising at least one alignment marker for orientation of the device with respect to a cavity.

66. A delivery device as claimed in claim 54 wherein the barrel has a greater cross-sectional area at its proximal end than at its distal end, whereby in use passage of an implant along the barrel in a distal direction in response to a force on the implant causes deformation of the implant.

67. A delivery device as claimed in claim 66 wherein at least a section of the barrel is inwardly tapered.

68. A delivery device as claimed in claim 54 wherein the barrel comprises a distal tip having a smaller cross-sectional area than the bore of the barrel whereby in use passage of an implant through the distal tip in response to a force on the implant causes deformation of the implant as it is discharged from the device.

69. A delivery device as claimed in claim 68 wherein the distal tip defines an adjustable aperture.

70. A delivery device as claimed in claim 54 wherein the device comprises a handle.

71. The delivery device of claim 54 further comprising a medical implant for implantation into a cavity.

72. Use of a delivery device as claimed in claim 54 for implanting a medical implant into a cavity.

73. A kit of parts comprising a delivery device as claimed in claim 54 and further comprising at least one of: at least one medical implant, at least one delivery device, at least one obturator, at least one surgical drill device, at least one surgical drill tip and instructions for a user.

74. A process for discharging a medical implant from the barrel of a delivery device as claimed in claim 56, the process comprising:
(i) applying a force to an implant to move the implant along the barrel in a distal direction wherein the movement causes deformation of the implant; and
(ii) applying further force to the implant to move the implant further along the barrel in a distal direction and discharge the deformed implant out of the barrel of the device.

75. A process as claimed in claim 74 wherein the force and further force are applied in separate sequential steps.

76. A process as claimed in claim 74 wherein the force and further force are applied in a single step.

77. A process as claimed in claim 74 wherein in response to a user actuating the implant contacting means, the implant contacting means applies a force to the implant to move the implant along the barrel in a distal direction.

78. A process as claimed in claim 74 wherein in response to a user actuating the plunger, the plunger applies the further force to move the implant further along the barrel in a distal direction and discharge the deformed implant out of the barrel of the device.

79. A process as claimed in claim 74 wherein in response to a user actuating the implant contacting means, the arms of the implant contacting means applies the further force to move the implant further along the barrel in a distal direction and discharge the deformed implant out of the barrel of the device.

80. A method of implanting a medical implant into a cavity in a patient, the method comprising:
(i) providing access to the cavity or hole through a surgical incision in the body of the patient;
(ii) discharging an implant by the method as claimed in any one of claims 74.
